(19) United States
(12) Patent Application Publication
(10) Pub. No.: US 2021/0000750 A1
(43) Pub. Date: Jan. 7, 2021

(54) ENGINEERED NANOVESICLES AS CHECKPOINT BLOCKADE FOR CANCER IMMUNOTHERAPY

(71) Applicant: NORTH CAROLINA STATE UNIVERSITY, Raleigh, NC (US)

(72) Inventors: Zhen GU, Los Angeles, CA (US); Xudong ZHANG, Raleigh, NC (US); Yanqi YE, Raleigh, NC (US)

(21) Appl. No.: 16/969,740

(22) PCT Filed: Feb. 15, 2019
(86) PCT No.: PCT/US2019/018208

§ 371 Date: Aug. 13, 2020

Related U.S. Application Data
(60) Provisional application No. 62/630,956, filed on Feb. 15, 2018.

(51) Int. Cl. A61K 9/50; A61K 9/51; C07K 16/28; A61K 31/675; A61P 35/00; A61K 47/68; A61K 47/69

(52) U.S. Cl. CPC A61K 9/5068; A61K 9/51; C07K 16/2827; A61K 2039/505; A61P 35/00; A61K 47/6851; A61K 47/6901; A61K 31/675

(57) ABSTRACT

Disclosed are engineered nanovesicles and engineered platelets comprising an exogenous protein and methods for treating cancer comprising administering the same to a subject.

This invention was made with government support under Grant No. 1L1TR001111 awarded by the National Institutes of Health. The government has certain rights in the invention. This application claims the benefit of U.S. Provisional Application No. 62/630,956, filed on Feb. 15, 2018 which is incorporated herein by reference in its entirety.

### I. BACKGROUND

Surgery is the main option for most solid tumors in clinical treatment. However, surgery often suffers the risk of relapse because of the incomplete resection of tumors. Furthermore, it has also been indicated that a surgery sometimes can promote cancer metastasis. Hence, there has been tremendous interest in developing effective strategies to treat cancer or prevent cancer relapse after surgery. Cancer immunotherapy aims to leverage the human immune system to eliminate cancer cells. Promisingly, tumor antigen specific T cells can eradicate the residual tumor cells. CD8⁺ T cells is one of the most important lymphocytes response to the tumor, especially that harbor the mutant genes. Indeed, these neoantigen (mutant protein derived antigens) specific CD8⁺ T cells can infiltrate into the tumor with positive immunotherapy outcome. However, programmed death-ligand 1 (PD-L1) expression in tumors suppresses T cells response and causes the T cells exhausted (Tₑₓ). Tₑₓ cells restrained by PD-L1 ligands through the inhibitory receptors programmed death-1 (PD-1). In addition, Tₑₓ cells disable the production of immune cytokines such as IFN-γ, TNF-α, granzyme B and perforin which leading fail to eradicate tumors. Blocking the PD-1/PD-L1 axis by checkpoint antibodies can reinvigorate Tₑₓ cells in clinical treatment and exhibit positive response to many types of human cancers, especially for melanoma. Checkpoint antibody therapy achieved rates of ˜37 to 38% in patients with melanoma, and similar response rates in other types of cancers such as renal cell carcinoma, non-small cell lung cancer and bladder cancer. However, anti-PD-1 therapy is not effective against all types of cancer. In fact, more than half patients showed resistance to the PD-1 antibody therapy, and only a minority of patients benefit from the treatment due to the multiple immune blockades. Meanwhile, most of the available humanized antibodies are produced from mice, which require complicated design and isolation. As a result, the cost of checkpoint antibody therapy remains unaffordable for many patients. Therefore, alternative approaches antagonizing the PD-1/PD-L1 inhibitor axis need to be developed.

### II. SUMMARY

Disclosed are methods and compositions related to engineered nanovesicle or engineered platelet encoding one or more exogenous protein receptors which can be used as checkpoint blockade in cancer immunotherapy.

In one aspect, the one or more exogenous protein receptors can comprise PD-1, TIGIT, LAG3, or TIM3.

Also disclosed herein are engineered nanovesicles or engineered platelets of any preceding aspect, wherein the engineered nanovesicles or engineered platelets is derived from a dendritic cell, stem cell, immune cell, megakaryocyte progenitor cell, or macrophage.

In one aspect, disclosed herein are pharmaceutical compositions comprising the engineered nanovesicles or engineered platelets of any preceding aspect.

Also disclosed herein are pharmaceutical compositions of any preceding aspect further comprising a therapeutic agent such as, for example, a small molecule (including, but not limited to 1-methyl-tryptophan (1-MT), norharmane, rosmarinic acid, epacadostat, navooximod, doxorubicin, tamoxifen, paclitaxel, vinblastine, cyclophosphamide, and 5-fluorouracil), siRNA, peptide, peptide mimetic, or antibody (such as, for example, and anti-PDL-1 antibody including, but not limited to Atexolizumab, Durvalumab, and Avelumab).

In one aspect, disclosed herein are pharmaceutical compositions of any preceding aspect, wherein the therapeutic agent is encapsulated in the engineered nanovesicle or engineered platelet.

Also disclosed herein are methods of treating a cancer (including, but not limited to melanoma, renal cell carcinoma, non-small cell lung carcinoma, and/or bladder cancer) in a subject comprising administering to a patient with a cancer the engineered nanovesicle, engineered platelets, or pharmaceutical composition of any preceding aspect.

In one aspect, disclosed herein are methods of treating a cancer in a subject of any preceding aspect, wherein the engineered nanovesicles, engineered platelets, or pharmaceutical composition are administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

Also disclosed herein are methods of treating a cancer in a subject of any preceding aspect, wherein the engineered nanovesicles, engineered platelets, or pharmaceutical composition are administered at least 1, 2, 3, 4, 5, 6, 7 times per week.

In one aspect, disclosed herein are methods of treating a cancer in a subject of any preceding aspect, wherein the dose of the administered engineered nanovesicle, engineered platelets, or pharmaceutical composition is from about 10 mg/kg to about 100 mg/kg.

Also disclosed herein are methods of treating a cancer in a subject of any preceding aspect, further comprising administering a chemotherapeutic agent.

Also disclosed herein are methods of treating a cancer in a subject of any preceding aspect, wherein the engineered nanovesicles, engineered platelets, or pharmaceutical composition are administered following surgical rescission of the tumor.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, 1H, and 1I show a schematic illustration and characterization of PD-1 blockade NVs for cancer immunotherapy. FIG. 1A shows a schematic illustration shows the preparation of PD-1 NVs loaded with 1-MT. (i) Engineering of HEK 293T cell line stably expressing mouse PD-1 receptors on the cell membranes. (ii) Harvesting of the cell membrane expressing PD-1 receptors. (iii) Preparation of PD-1 NVs through extrusion. FIG. 1B shows that PD-L1 exhausts CD8⁺ T cells by interacting with PD-1 receptors. The expression of IDO is induced by Treg cells, which inhibits the activity of CD8⁺ T cells. FIG. 1C shows PD-L1 blockade by PD-1 NVs reverts the exhausted CD8⁺ cells to attack tumor cells. The release of IDO inhibitor 1-MT also reverts the exhausted CD8⁺ T cells. FIG. 1D shows the establishment of HEK 293T cell line stably expressing mouse PD-1 on cell membranes. WGA Alexa-Fluor 488 dye was used to label cell membrane. Scale bar: 10 μm. FIG. 1E shows the TEM image showed the shape and size of PD-1 NVs. Scale bar: 100 nm. FIG. 1F shows frozen scanning electron microscopy (SEM) image showed the natural shape of the PD-1 NVs (Scale bar: 100 nm). FIG. 1G shows the confocal image indicated the existence of DsRed-PD-1 NVs by the red spots. Scale bar: 1 μm. FIG. 1H shows the size distribution of PD-1 NVs measured by DLS. FIG. 1I shows western blot assay exhibited the expression of mouse PD-1 receptors on the NVs and whole cell lysis (HCLs) of the stable cell line. Na⁺ K⁺ ATPase was used as loading control.

FIGS. 2A, 2B, and 2C show characterization of PD-1 MVs. FIG. 2A shows the confocal images indicate the existence of DsRed-PD-1 MVs by the red spots. Scale bar: 1000 nm. FIG. 2B show the size distribution of PD-1 MVs measured by DLS. FIG. 2C show the zeta potential of free NVs and PD-1 NVs (n=3). Error bar, mean±s.d.

FIGS. 3A, 3B, 3C, 3D, 3E, 3F, 3G, 3H, and 3 show in vitro biological behavior and in vivo biodistribution of PD-1 NVs. FIG. 3A shows DsRed-PD-1 NVs bound on the cell membrane of B16F10 cancer cells. PD-1 NVs (50 μg/mL) or PD-1 free NVs labeled with Cy5.5 (50 μg/mL) were incubated with B16F10 cells for 2 h. WGA Alexa-Fluor 488 dye was used to detect B16F10 cell membrane (Scar bar: 10 μm). FIG. 3B shows DsRed-PD-1 NVs were incepted by DCs. PD-1 NVs (50 μg/mL) were incubated with DCs for 2 h. WGA Alexa-Fluor 488 dye was used to detect DC membrane. Scar bar: 10 μm. FIG. 3C shows B16F10 cells were transfected with EGFP-PD-L1 plasmid for 20 h, then incubated with PD-1 NVs (50 μg/mL) for 5 h, the co-localization of PD-1 NVs and PD-L1 proteins was detected (Scar bar: 10 μm). The above images are the enlarged ones in the white collar on the underside images. FIG. 3D shows the representative flow cytometric analysis images of PD-1 NVs binding with B16F10 cells (gated on DsRed⁺). PD-1 NVs (50 μg/mL) were incubated with B16F10 cells for 2 h. Or aPD-L1 antibody (20 μg/mL) were incubated with the cells for 4 h before the PD-1 NVs were added in the culture medium as indicated. FIG. 3E shows CO-IP and western blot were used to examine the interaction between PD-1 (on NVs) and PD-L1 (on B16F10 cells). FIG. 3F shows Cy5.5 labeled free NVs (200 μL, 5 mg/mL) and PD-1 NVs (200 μL, 5 mg/mL) were injected through tail-vein of the mice. Fluorescence was measured at different time points as indicated (n=3) Error bar, mean±s.d. FIG. 3G shows the IVIS spectrum images of distribution of free NVs and PD-1 NVs in tumor and major organs. Left: lung, heart and liver. Right: spleen, kidney and tumor. FIG. 3H shows the fluorescence intensity per gram of tissue in tumor and major organs as indicated (n=3). Error bar, mean±s.d. FIG. 3I show the distribution of PD-1 NVs in the organs and tumor sections were detected using confocal microscope. Scar bar: 100 μm.

FIGS. 4A and 4B show in vivo anti-tumor effect of PD-1 NVs with different dosage through the tail-vein injection. FIG. 4A shows in vivo bioluminescence imaging of the B16F10 melanoma tumors. FIG. 4B shows average tumor volumes of mice with different treatments (n=7). Error bar, mean±s.e.m. NS: no significant, *P<0.05, **P<0.01, ***P<0.001; by one-way analysis of variance (ANOVA) with Tukey post-hoc tests.

FIGS. 5A, 5B, 5C, 5D, 5E, 5F, 5G, 5H, 5I, and 5J show the In vivo anti-tumor effect of PD-1 NVs. FIG. 5A shows in vivo bioluminescence imaging of the B16F10 melanoma tumor of different mice groups at different time points after the tail-vein injection of free NVs, PD-1 NVs and PD-L1 antibody. Day 0: the day for the first time of treatment. FIG. 5B shows average tumor volumes of the treated mice in different groups (n=7). Error bar, mean±s.e.m. FIG. 5C shows images of representative tumors extracted from euthanized mice of different groups (n=7). Error bar, mean±s.d. FIG. 5D shows survival curves for the mice received the treatment of PD-1 NVs, PD-L1 antibody and free NVs (n=10). FIG. 5E shows body weights of mice received the treatment and control mice. Error bar, mean±s.d. FIG. 5F shows IFN-γ levels in serum from mice isolated at day 20 after mice received the first indicated treatment (n=3). Error bar, mean±s.d. FIGS. 5G and 5H show representative plots (5F) and quantitative analysis (5G) of T cells (gated on CD3+ cells) in treated tumor analyzed by flow cytometry (n=3). Error bar, mean±s.d. FIGS. 5I and 5J show representative image (5I) and quantitative analysis (5J) of immunofluorescence staining of the tumor sections showing infiltrated CD4+ T cells and CD8⁺ T cells (n=3). Error bar, mean±s.d. Scar bar: 100 μm. Throughout, NS: no significant, *P<0.05, **P<0.01, ***P<0.001; by one-way analysis of variance (ANOVA) with Tukey post-hoc tests (5B, 5C, 5F, 5H, 5J) or by Log-Rank (Mantel-Cox) test (5D).

FIG. 6 shows IDO enzyme activity was measured as the inhibition of kynurenine production after treatment of the free 1-MT, 1-MT@NVs and 1-MT@PD-1 NVs.

FIG. 7 shows Fluorescence intensity per gram of tumor tissues at different time point as indicated (n=3). Error bar, mean±s.d.

FIG. 8 shows in vivo suppression of tumor growth by 1-MT-loaded PD-1 NVs. FIG. 8A shows In vivo bioluminescence imaging of the B16F10 tumor of the mice received different treatments: PBS (Group 1), free NVs (Group 2), 1-MT (Group 3), PD-1 NVs (Group 4), 1-MT@ NVs (Group 5), 1-MT+ aPD-L1 (Group 6), 1-MT @ PD-1 NVs (Group 7). Day 0: the day for the first time of treatment. FIG. 8B shows the average tumor volumes of the treated mice in different groups as indicated (n=7). Error bar, mean±s.e.m. FIG. 8C shows survival curves for the mice received different treatment as indicated (n=10). FIGS. 8D and 8E show representative flow cytometry plots (8D) and quantitative analysis (dE) of T cells in the tumors from different treatment groups (n=3). The cells were pre-gated for positive CD3⁺ expression. Error bar, mean±s.d. FIG. 8F shows immunofluorescence of the tumors showed infiltrated CD4+ T cells and CD8⁺ T cells. Scar bar: 100 μm. Throughout, NS: no significant, *P<0.05, **P<0.01, ***P<0.001; two two-way ANOVA analyses were carried out to do the analyses (8B and 8E). First two-way ANOVA with Tukey post-hoc test analysis carried out between the group of Free-NVs (G2), PD-1 NVs (G4), 1-MT@NVs (G5) and 1-MT@PD1-NVs (G7). The two factors considered were 1-MT and PD-1. The second two-way ANOVA with Tukey post-hoc test carried out between the groups of the PBS control (G1), aPD-L1, 1-MT (G3) and aPD-L1+1-MT (G6). The two factors in this model were 1-MT and aPD-L1 (8B and 8E) or by Log-Rank (Mantel-Cox) test (8C).

FIGS. 9A, 9B, and 9C show schematic of the production of PD-1-expressing platelets and reinvigoration of CD8⁺ T cells. FIG. 9A shows a schematic shows L8057 cell lines stably expressing murine PD-1 and production of platelets. FIG. 9B shows that PD-1-expressing platelets target tumor cells within the surgery wound. FIG. 9C shows that PD-L1 blockade by PD-1-expressing platelets reverts exhausted CD8⁺ T cells to attack tumor cells.

FIGS. 10A, 10B, 10C, 10D, 10E, 10F, 10G, 10H, 10I, 10J, and 10K show production and characterization of platelets from PD-1-expressing L8057 stable cell lines. FIG. 10A shows a confocal image present L8057 cell lines stably expressing murine EGFP-PD-1 on cell membranes. WGA Alexa-Fluor 594 dye was used to stain cell membrane (Scale bar: 10 μm). FIG. 10B shows western blot analysis for evaluating the expression of PD-1 in L8057 cell line. L8. is short for L8057 cells. FIG. 10C shows EGFP-PD-1-expressing L8057 cells stimulated with 500 nM PMA for 3 days, and immunostained to detect CD42a expression. FIG. 10D shows L8057 cells stimulated with 500 nM PMA for 3 days, and stained with Wright-Giemsa dye (Scale bar: 10 μm). FIG. 10E shows the evolution process of PD-1-expressing proplatelet extended from MKs (Scale bar: 10 μm). FIG. 10F shows the morphology of PD-1 proplatelets extended from L8057 cells after 6 days of stimulation with 500 nM PMA. PD-1 proplatelets extended from L8057 cells (Scale bar: 10 μm). FIG. 10G shows representative confocal images of purified PD-1-expressing platelets (Scale bar: 10 μm). FIG. 10H shows size distribution of PD-1-expressing platelets measured by DLS. FIG. 10I shows CSEM image shows the morphology of PD-1-expressing platelets (Scale bar: 1 μm). FIG. 10J shows representative TEM image shows morphology and size of PD-1-expressing platelet (Scale bar: 1 μm). FIG. 10K shows the number of platelets released from PD-1-expressing L8057 cells after stimulated with 500 nM PMA (n=5).

FIGS. 11A, 11B, 11C, 11D, 11E, 11F, 11G, 11H, and 11I show the in vitro and in vivo biobehavior of PD-1 platelets. FIGS. 11A and 11B show retention of platelets on collagen-coated or un-coated tissue culture slides. Scale bar, 50 μm. FIG. 11C shows confocal, CSEM and TEM images of PD-1 platelets stimulated with thrombin. Platelet microparticles (PMPs) were released from the platelets. FIG. 11D shows measurement of the size distribution of PD-1 platelets after activation by the treatment with thrombin for 30 min. PMPs were produced from the platelets. FIG. 11E shows EGFP-PD-1 platelets bound on the cell membrane of B16F10 cells. PD-1 platelets or free platelets labeled with Cy5.5 were incubated with B16F10 cells for 20 h. WGA Alexa-Fluor 594 dye was used to stain the B16FI cell membrane (Scar bar: 10 μm). FIG. 11F shows B16F10 cells that were transfected with DsRed-PD-L1 plasmid for 20 h, then incubated with EGFP-PD-1 platelets for 5 h, the co-localization of EGFP-PD-1 platelets and DsRed-PD-L1 was detected (Scar bar: 10 μm). FIG. 11G shows Cy5.5 labeled free platelets and PD-1 platelets were injected through tail-vein of the mice. Fluorescence was measured at different time points as indicated (n=3). Error bar, mean±s.d. FIG. 11H shows in vivo fluorescence images of distribution of free platelets and PD-1 platelets in major organs and residual tumor. FIG. 11I shows fluorescence intensity per gram of tissue in major organs and tumors as indicated (n=3). Error bar, mean±s.d.

FIGS. 12A, 12B, 12C, 12D, 12E, 12F, 12G, 12H, and 12I show that PD-1 platelets suppress the tumor progress in incomplete-surgery tumor model. FIG. 12A shows a schematic illustration of PD-1 platelets used for therapy in an incomplete-surgery tumor model. FIG. 12B shows in vivo bioluminescence imaging of the B16F10 tumor from surgical mice received different treatment: PBS, free platelets, and PD-1 platelets, respectively. FIG. 12C shows the average tumor volumes of the treated mice in different group as indicated. Data are shown as the mean±s.e.m. FIG. 12D shows the survival curves for the mice received different treatments as indicated. FIG. 12E shows the immunofluorescence of the tumors sections showed CD4⁺ T cells and CD8⁺ T cells infiltration (Scar bar: 100 μm). FIGS. 12F and 12G show representative plots (12F) and quantitative (12G) of T cells in tumors of different treatment groups analyzed by the flow cytometry (Gated on CD3⁺ T cells). FIGS. 12H and 12I show representative plots (12H) and quantitative (12I) of GzmB in CD8⁺ T cells of the tumors in different treatment groups analyzed by the flow cytometry (Gated on CD8⁺ T cells). Throughout, NS: no significant, *P<0.05, **P<0.01, ***P<0.001; one-way ANOVA with Tukey post-hoc test analyses were carried out to do the analyses (12C, 12G, and 12I) or by Log-Rank (Mantel-Cox) test (12D).

FIGS. 13A, 13B, 13C, 13D, 13E, 13F, 13G, 13H, 13I, 13J, and 13K shows in vivo antitumor effect of cyclophosphamide-loaded PD-1-expressing platelets in incomplete-surgery tumor model. FIG. 13A shows average tumor volumes of mice (n=8) treated with: PBS (G1), cyclophosphamide (CP) (G2), PD-1-expressing platelets (G3), CP-free platelets (G4), and CP-loaded PD-1-expressing platelets (G5). Data are shown as the mean±s.e.m. Compared with PBS control. FIG. 13 shows survival curves of the treated mice. FIG. 13C shows quantification of FoxP3 expression in CD4⁺ T cells within the tumors analyzed by the flow cytometry (gated on CD4⁺ T cells) (n=3). FIGS. 13D and 13E show representative plots (13D) and quantification (13E) of Ki67 in CD8⁺ T cells within the tumors analyzed by the flow cytometry (gated on CD8⁺ T cells) (n=3). FIGS. 13F and 13G show representative plots (13F) and quantification (13G) of CD8⁺ and CD4⁺ T cells within tumors analyzed by the flow cytometry (gated on CD3⁺ T cells) (n=3). FIGS. 13H and 13 show representative plots (13H) and quantification (13I) of GzmB in CD8⁺ T cells within the tumors analyzed by the flow cytometry (gated on CD8⁺ T cells) (n=3). FIGS. 13J and 13K show immunofluorescence of the tumors showing CD8⁺ T cell infiltration (Scale bar: 100 μm). Throughout, NS: no significant, *P<0.05, **P<0.01, ***P<0.001; two-way ANOVA with Tukey post-hoc test analyses were carried out to do the analyses (13A, 13C, 13E, 13G, 13I, 13K) or by Log-Rank (Mantel-Cox) test (13B).

FIGS. 14A, 14B, and 14C show B16F10 tumor growth in mice treated with PD-1-expressing platelets after partial tumor resection. FIG. 14A shows In vivo tumor bioluminescence of B16F10 tumors. FIG. 14B shows representative plots of FoxP3 expression in CD4⁺ T cells within tumors analyzed by the flow cytometry (gated on CD4⁺ T cells) (n=3). FIG. 14C shows the body weights of treated and control mice. Error bar, ±s.d.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

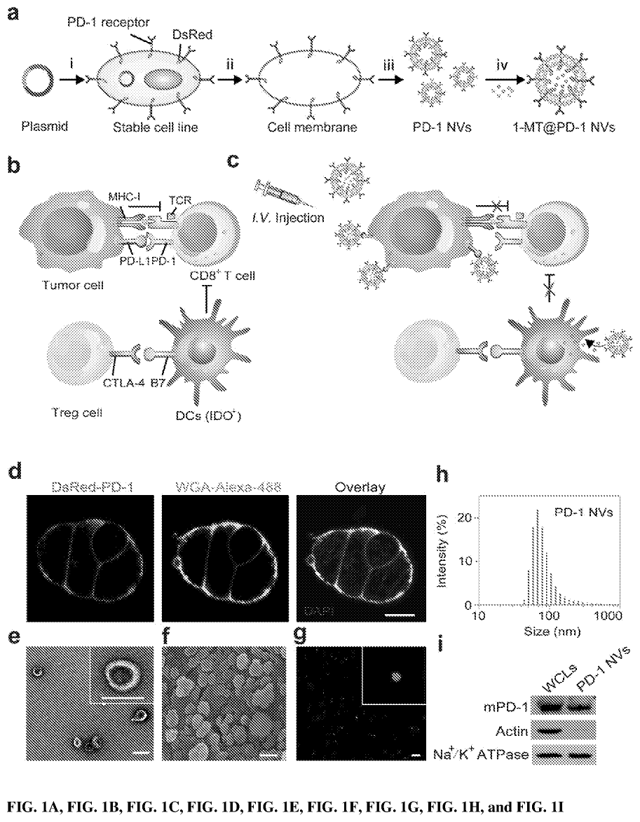

It is understood and herein contemplated that immunotherapy such as checkpoint inhibitor blockade can be effective in the treatment of cancers or relapse following surgical recision of a tumor. However, the antibodies used in these blockades result in limitations for many patients and are ineffective in many more. Disclosed herein, natural cell membrane derived vesicles such as exosomes, macrovesicles and cell membrane excluded vesicles hold great promise for biomedicine. Similarly, bioengineering strategies as promising ways for the enhancement of anticancer immunity. Herein, cell membrane derived nanovesicles (NVs) were engineered to display PD-1 receptors, which enhance the cancer immunotherapy through disrupting disturbing the PD-1/PD-L1 immune inhibitory axis (FIG. 1a). similarly, engineered conjugated with anti-PD-L1 can target tumor surgery woulds to reninvigorate exhausted T cells. Accordingly, in on aspect, disclosed herein are engineered nanovesicle, engineered megakaryocytes, or engineered platelets encoding one or more exogenous protein receptors which can be used as checkpoint blockade in cancer immunotherapy.

As noted above, the blockade of immune inhibitory interactions can rescue or prevent T cell exhaustion and allow the immune system to eliminate a tumor and prevent tumor proliferation and/or metastasis alone or following surgical recision. There are several important immune system blockades known in the art including program death 1 (PD-1)/program death ligand 1 (PDL-1); T cell immunoreceptor with Ig and ITIM domains (TIGIT)/CD155; T-cell immunoglobulin and mucin-domain containing-3 (TIM-3)/galectin-9, phospatidyl serine (PtdSer), Carcinoembryonic Antigen Related Cell Adhesion Molecule 1 (CEACAM1), or High Mobility Group Protein 1 (HMGB1); and/or lymphocyte-activation gene 3 (LAG3)/MHC-class II. Accordingly, in one aspect, disclosed herein are engineered nanovesicle, engineered megakaryocytes, or engineered platelets encoding one or more exogenous protein receptors which can be used as checkpoint blockade in cancer immunotherapy, wherein the one or more exogenous protein receptors can comprise PD-1, TIGIT, LAG3, and/or TIM3.

It is understood and herein contemplated that the engineered nanovesicles, engineered megakaryocytes, or engineered platelets can be derived from any cell that can support their manufacture, including but not limited to dendritic cells, stem cells, immune cells, megakaryocyte progenitor cells, megakaryocytes, or macrophages. Accordingly, in one aspect, disclosed herein are engineered nanovesicle, engineered megakaryocytes, or engineered platelets encoding one or more exogenous protein receptors which can be used as checkpoint blockade in cancer immunotherapy, wherein the engineered nanovesicles, engineered megakaryocytes, or engineered platelets is derived from a dendritic cell, stem cell, immune cell, megakaryocyte progenitor cell, or macrophage.

1. Pharmaceutical Carriers/Delivery of Pharmaceutical Products

In one aspect, it is understood that the engineered nanovesicles, engineered megakaryocytes, or engineered platelets disclosed herein are intended for administration to a subject to treat, prevent, inhibit, or reduce a cancer or metastasis or to treat, prevent, inhibit, or reduce a relapse or metastasis following surgical recision (i.e., resection). Thus, disclosed herein are pharmaceutical compositions comprising the engineered nanovesicles, engineered megakaryocytes, or engineered platelets disclosed herein. For example disclosed herein are pharmaceutical compositions comprising engineered nanovesicles, engineered megakaryocytes, or engineered platelets encoding one or more exogenous protein receptors which can be used as checkpoint blockade in cancer immunotherapy, wherein the one or more exogenous protein receptors can comprise PD-1, TIGIT, LAG3, or TIM3.

In one aspect, it is understood and herein contemplated that other inhibitors of other immunomodulatory pathways can have additional benefits to the treatment of a cancer in combination with the disclosed engineered nanovesicles, engineered megakaryocytes, and engineered platelets. For example, inhibitor of Indoleamine 2,3-dioxygenase (IDO) with, for example, 1-methyl-tryptophan (1-MT), can enhance the immune response to a cancer. Similarly, anti-PDL-1 antibodies (such as, for example, and anti-PDL-1 antibody including, but not limited to nivolumab, pembrolizumab, pidilizumab, BMS-936559, Atexolizumab, Durvalumab, and Avelumab) could bind any PDL-1 that the engineered nanovesicles, engineered megakaryocytes, or engineered platelets fail to bind. Accordingly, disclosed herein are pharmaceutical compositions comprising engineered nanovesicles, engineered megakaryocytes, or engineered platelets encoding one or more exogenous protein receptors which can be used as checkpoint blockade in cancer immunotherapy, wherein the one or more exogenous protein receptors can comprise PD-1, TIGIT, LAG3, or TIM3 further comprising one or more therapeutic agents such as, for example, a small molecule (including, but not limited to 1-methyl-tryptophan (1-MT), norharmane, rosmarinic acid, epacadostat, navooximod, doxorubicin, tamoxifen, paclitaxel, vinblastine, cyclophosphamide, and 5-fluorouracil), siRNA, peptide, peptide mimetic, or antibody (such as, for example, and anti-PDL-1 antibody including, but not limited to nivolumab, pembrolizumab, pidilizumab, BMS-936559, Atexolizumab, Durvalumab, and Avelumab).

The one or more therapeutic agents can be provided in the pharmaceutical composition along with the engineered nanovesicles, engineered megakaryocytes, or engineered platelets. Alternatively, the one or more therapeutic agent can be encapsulated in the engineered nanovesicles, engineered megakaryocytes, or engineered platelets. Thus, in one aspect, disclosed herein are pharmaceutical compositions comprising engineered nanovesicles, engineered megakaryocytes, or engineered platelets encoding one or more exogenous protein receptors which can be used as checkpoint blockade in cancer immunotherapy, wherein the one or more exogenous protein receptors can comprise PD-1, TIGIT, LAG3, or TIM3 further comprising one or more therapeutic agent such as, for example, a small molecule (including, but not limited to 1-methyl-tryptophan (1-MT), norharmane, rosmarinic acid, epacadostat, navooximod, doxorubicin, tamoxifen, paclitaxel, vinblastine, cyclophosphamide, and 5-fluorouracil), siRNA, peptide, peptide mimetic, or antibody (such as, for example, and anti-PDL-1 antibody including, but not limited to nivolumab, pembrolizumab, pidilizumab, BMS-936559, Atexolizumab, Durvalumab, and Avelumab); and wherein the one or more therapeutic agents are encapsulated in the engineered nanovesicles, engineered megakaryocytes, or engineered platelets.

As the disclosed pharmaceutical compositions comprising the disclosed engineered nanovesicles, engineered megakaryocytes, or engineered platelets can be used to treat cancer it is further contemplated therein that the disclosed pharmaceutical compositions can further comprise any known any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. 56. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

2. Method of Treating Cancer

As noted herein, the disclosed engineered nanovesicles, engineered megakaryocytes, engineered platelets, and/or pharmaceutical compositions can be used to treat any disease where uncontrolled cellular proliferation occurs such as cancers. Accordingly, in one aspect, disclosed herein are methods of treating, reducing, inhibiting, or preventing a cancer (including, but not limited to melanoma, renal cell carcinoma, non-small cell lung carcinoma, and/or bladder cancer); proliferation of a cancer (including, but not limited to melanoma, renal cell carcinoma, non-small cell lung carcinoma, and/or bladder cancer); metastasis of a cancer (including, but not limited to melanoma, renal cell carcinoma, non-small cell lung carcinoma, and/or bladder cancer); and/or treating, reducing, inhibiting, or preventing relapse, proliferation or metastasis of a cancer following surgical recision of a tumor (including, but not limited to melanoma, renal cell carcinoma, non-small cell lung carcinoma, and/or bladder cancer) in a subject comprising administering to a patient with a cancer the engineered nanovesicle, engineered magekaryocytes, engineered platelets, and/or pharmaceutical composition disclosed herein. Thus, in one aspect, disclosed herein are methods of treating, reducing, inhibiting, or preventing a cancer; proliferation of a cancer; metastasis of a; and/or treating, reducing, inhibiting, or preventing relapse, proliferation or metastasis of a cancer following surgical recision of a tumor in a subject comprising administering to a subject engineered nanovesicles, engineered megakaryocytes, or engineered platelets encoding one or more exogenous protein receptors which can be used as checkpoint blockade in cancer immunotherapy (or a pharmaceutical composition comprising the same), wherein the one or more exogenous protein receptors can comprise PD-1, TIGIT, LAG3, or TIM3. It is understood that the engineered nanovesicles, engineered megakaryocytes, engineered platelets, and/or pharmaceutical compositions used in the disclosed methods can further comprise one or more therapeutic agents to enhance the immunotherapeutic effect of the engineered nanovesicles, engineered megakaryocytes, engineered platelets, and/or pharmaceutical composition. For example, the engineered nanovesicles, engineered megakaryocytes, engineered platelets, and/or pharmaceutical compositions used in the disclosed methods can further comprise a small molecule (including, but not limited to 1-methyl-tryptophan (1-MT), norharmane, rosmarinic acid, epacadostat, navooximod, doxorubicin, tamoxifen, paclitaxe, vinblastine, cyclophosphamide, and 5-fluorouracil), siRNA, peptide, peptide mimetic, or antibody (such as, for example, and anti-PDL-1 antibody including, but not limited to nivolumab, pembrolizumab, pidilizumab, BMS-936559, Atexolizumab, Durvalumab, and Avelumab). The one or more therapeutic agents can be encapsulated in the engineered nanovesicles, engineered megakaryocytes, and/or engineered platelets or supplied in the pharmaceutical composition along with the engineered nanovesicles, engineered megakaryocytes, and/or engineered platelets. Accordingly, disclosed herein are methods of treating, reducing, inhibiting, or preventing a cancer; proliferation of a cancer; metastasis of a; and/or treating, reducing, inhibiting, or preventing relapse, proliferation or metastasis of a cancer following surgical recision of a tumor in a subject comprising administering to a subject engineered nanovesicles, engineered megakaryocytes, or engineered platelets encoding one or more exogenous protein receptors which can be used as checkpoint blockade in cancer immunotherapy (or a pharmaceutical composition comprising the same), wherein the one or more exogenous protein receptors can comprise PD-1, TIGIT, LAG3, or TIM3; and wherein the engineered nanovesicles, engineered megakaryocytes, engineered platelets, and/or pharmaceutical compositions further comprise a small molecule (including, but not limited to 1-methyl-tryptophan (1-MT), norharmane, rosmarinic acid, epacadostat, navooximod, doxorubicin, tainoxifen, paclitaxel, vinblastine, cyclophosphamide, and 5-fluorouracil), siRNA, peptide, peptide mimetic, or antibody (such as, for example, and anti-PDL-1 antibody including, but not limited to nivolumab, pembrolizumab, pidilizumab, BMS-936559, Atexolizumab, Durvalumab, and Avelumab).

It is understood and herein contemplated that the chemotherapeutic used in the disclosed cancer treatment, inhibition, reduction, and/or prevention methods can comprise any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritale (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Accordingly, disclosed herein are methods of treating, reducing, inhibiting, or preventing a cancer; proliferation of a cancer; metastasis of a; and/or treating, reducing, inhibiting, or preventing relapse, proliferation or metastasis of a cancer following surgical recision of a tumor in a subject comprising administering to a subject engineered nanovesicles, engineered megakaryocytes, or engineered platelets encoding one or more exogenous protein receptors which can be used as checkpoint blockade in cancer immunotherapy (or a pharmaceutical composition comprising the same), wherein the one or more exogenous protein receptors can comprise PD-1, TIGIT, LAG3, or TIM3; further comprising administering to the subject separately or in the same composition any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Said methods can also include the administration of any of the therapeutic agents disclosed herein including but not limited to a small molecule (including, but not limited to 1-methyl-tryptophan (1-MT), norharmane, rosmarinic acid, epacadostat, navooximod, doxorubicin, tamoxifen, paclitaxel, vinblastine, cyclophosphamide, and 5-fluorouracil), siRNA, peptide, peptide mimetic, or antibody (such as, for example, and anti-PDL-1 antibody including, but not limited to nivolumab, pembrolizumab, pidilizumab, BMS-936559, Atexolizumab, Durvalumab, and Avelumab).

As noted above, the disclosed methods or useful in the treatment of cancer. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer

In one aspect, the disclosed methods of treating a cancer comprising administering to a subject any of the engineered nanovesicles, engineered platelets, or pharmaceutical composition disclosed herein can comprise administration of the engineered nanovesicles, engineered platelets, or pharmaceutical composition at any frequency appropriate for the treatment of the particular cancer in the subject. For example, the engineered nanovesicles, engineered platelets, or pharmaceutical composition can be administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months. In one aspect, the engineered nanovesicles, engineered platelets, or pharmaceutical composition are administered at least 1, 2, 3, 4, 5, 6, 7 times per week.

In one aspect, the amount of the engineered nanovesicles, engineered platelets, or pharmaceutical composition administered to the subject for use in the disclosed methods can comprise any amount appropriate for the treatment of the subject for the particular cancer as determined by a physician. For example, the amount of the engineered nanovesicles, engineered platelets, or pharmaceutical composition can be from about 10 mg/kg to about 100 mg/kg. For example, the amount of the engineered nanovesicles, engineered platelets, or pharmaceutical composition administered can be at least 10 mg/k, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. Accordingly, in one aspect, disclosed herein are methods of treating a cancer in a subject, wherein the dose of the administered engineered nanovesicle, engineered platelets, or pharmaceutical composition is from about 10 mg/kg to about 100 mg/kg.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: PD-1 Blockade Cellular Vesicles for Cancer Immunotherapy

Natural cell membrane derived vesicles such as exosomes, macrovesicles and cell membrane excluded vesicles hold great promise for biomedicine. Similarly, bioengineering strategies as promising ways for the enhancement of anticancer immunity. Herein, cell membrane derived nanovesicles (NVs) were engineered to display PD-1 receptors, which enhance the cancer immunotherapy through disrupting the PD-1/PD-L1 immune inhibitory axis (FIG. 1A). The PD-1 NVs can bind to the surface of tumor cells and achieve PD-L1 blockade (FIGS. 1A, 1B, and 1C). This blockade is expected to effectively revert the exhausted tumor antigen-specific CD8⁺ to attack the tumor cells. In addition, the NVs can also serve as carriers for other therapeutics to perform combination delivery. Indoleamine 2,3-dioxygenase (IDO) is an immunosuppressive molecule overexpressed by tumor to limit the proliferation and function of effector T cells. Here 1-methyl-tryptophan (1-MT), a small molecule inhibitor of IDO, was encapsulated into PD-1 NVs to simultaneously block the PD-1/PD-L1 axis and overcome the inhibitory effects of tumor-associated IDO on effector T cells within the tumor microenvironment (TME) (FIG. 1C).

To prepare PD-1 NVs, HEK 293T cells were established that stably express the mouse PD-1 receptor on the cell membrane. HEK 293T cell line has been widely used in cell biology research and biotechnology industry because it can be robustly transfected and produces high amount of recombinant proteins. DsRed protein-tag was included in the C-terminal portion of PD-1 receptor protein, which made the protein-tag close to the inner leaflet of cell membranes, while the functional domain of the receptors is extracellular (FIG. 1a). Therefore, the mouse PD-1 receptor cDNA was cloned into a mammalian expression vector. The transfected HEK 293T cells were selected with hygromycin B to establish a stable cell line. Notably, the death receptor PD-1 was mainly expressed and localized on the cell membranes (FIG. 1d). Under the selection pressure of hygromycin B, the cell line continued to express DsRed PD-1 receptors for more than twenty passages. Furthermore, the cell membranes were labeled with Alexa-Fluor 488 conjugate wheat germ agglutinin (WGA) to confirm the localization of the PD-1 receptors. As expected, the red fluorescence of DsRed protein co-localized with green fluorescence of WGA Alexa-Fluor 488 dye on the cell membranes (FIG. 1d).

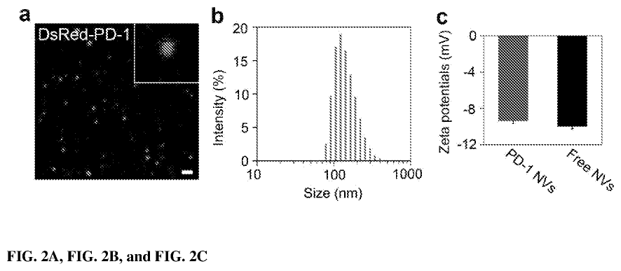

Next, the engineered HEK 293T cells were cultured and lysed to isolate the cell membranes. Cell membrane vesicles expressing PD-1 receptors were prepared by a serial extrusion of vesicles through 0.8 and 0.22 μm pore-sized polycarbonate membrane filters. After extrusion through the 0.8 μm pore-sized polycarbonate membrane filters, major cell membrane vesicles (MVs) were obtained. The red-light spots in the confocal image demonstrated the existence of DsRed-PD-1 on MVs (FIG. 2a). The size distribution of MVs was measured by dynamic light scattering (DLS) analysis (FIG. 2b). The MVs were then extruded through 0.22 μm pore-sized polycarbonate membrane filters. The harvested NVs were further purified by a two-step OptiPrep density gradient ultracentrifugation. Next, the morphology of the NVs was characterized by electron microscopy. Negatively stained NVs revealed that they were closed vesicles using transmission electron microscopy (TEM) (FIG. 1e). The NVs were also scanned by the frozen scanning electron microscopy (SEM), which showed that the NVs had a spherical shape (FIG. 1f). The zeta potential of the NVs was determined as −10 mV (FIG. 2c). Moreover, the expression of PD-1 receptors on the NVs was detected using confocal imaging and western blot. The confocal image exhibited the red-colored spots indicated the existence of DsRed-PD-1 NVs (FIG. 1g). DLS analysis showed that the average diameter of NVs was around 90-100 nm (FIG. 1h). Additionally, western blot analysis indicated that the purified NVs displaying the PD-1 receptors (FIG. 1i). To verify that whether the PD-1 receptors maintained an outside-out orientation on NV surfaces, an immunoprecipitation assay (IP) was performed. The assay showed that the PD-1 antibody pulled down the majority of PD-1 NVs, which demonstrated that PD-1 receptors have a correct outside-out orientation on most PD-1 NVs.

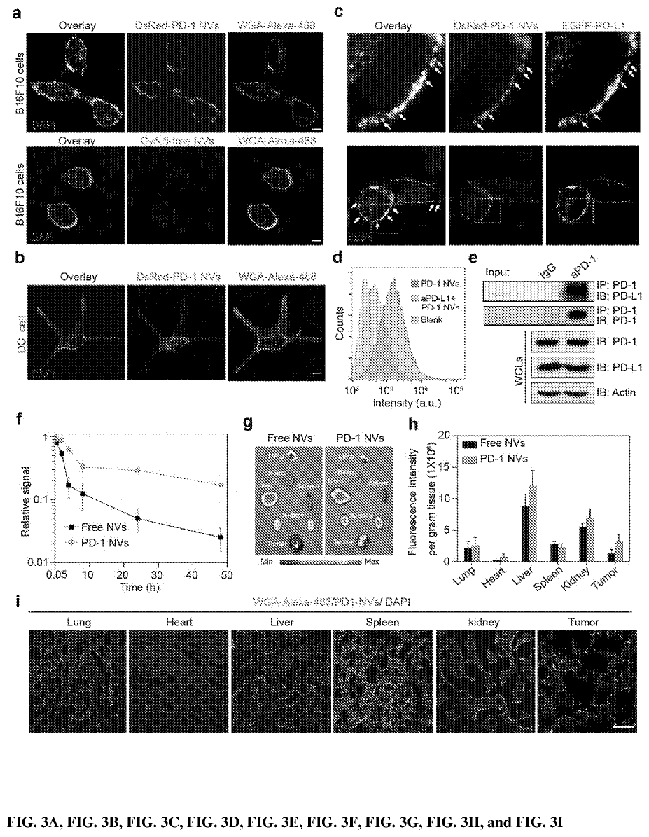

Cancer cells exhaust antigen-specific CD8⁺ T cells through overexpression of PD-L1 ligands that interact with PD-1 receptors. To investigate whether PD-1 NVs bind to melanoma cells, the PD-1 NVs were incubated with B16F10 melanoma cells in vitro. DsRed proteins fused with PD-1 receptors provided red fluorescence, which was used as a fluoresce signal label of the PD-1 NVs. WGA Alexa-Fluor 488 dye was used to stain the cell membranes of the B16F10 melanoma cells. Remarkably, it was observed that PD-1 NVs effectively bound around the cell membrane surface of B16F10 cells after incubation for 2 h (FIG. 3a). In contrast, Cy5.5 labeled the free NVs had low membrane binding affinity (FIG. 3a). In addition, the interaction between PD-1 NVs and dendritic cells (DCs) was also detected. PD-1 NVs were incubated with bone marrow-derived DCs (BMDCs) for 2 h. The confocal image showed that DsRed-PD-1 NVs can effectively bind and be internalized by the BMDCs after 2 h (FIG. 3b). To investigate whether the binding of PD-1 NVs on the B16F10 cells were through the interaction between PD-1 and PD-L1, the co-localization between PD-1 receptors on NVs and PD-L1 on B16F10 cells was firstly detected. PD-1 NVs were incubated with EGFP-PD-L1 expressing B16F10 cells for 5 h. Notably, PD-1 NVs were co-localized with EGFP-PD-L1 on the B16F10 melanoma cells (FIG. 3c). To confirm the molecular binding between PD-1 receptors on NVs and PD-L1 on the B16F10 cells anti-PD-L1 antibody was added to block the PD-L1 on the B16F10 cells. The confocal images shown that PD-1 NVs binding are dramatically reduced when PD-L1 antibody (aPD-L1) were pre-incubated with the cells. Moreover, the flow cytometric data also shown that the quantity of PD-1 NVs binding with B16F10 cells are significantly reduced when PD-L1 antibody were pre-incubated with the cells (FIG. 3d). A co-immunoprecipitation (CO-IP) assay as also employed to detect the molecular interaction between PD-1 receptor and PD-L1. After incubation of the PD-1 NVs with B16F10 melanoma cells for 20 h, the cells were harvested. PD-1 primary antibody was used to pull down the PD-1 receptors on the NVs. Remarkably, PD-L1 were pulled down together with PD-1 receptors by the PD-1 antibody (FIG. 3e), indicating that PD-1 NVs physically interact with PD-L1 expressed by B16F10 cells. Together, these results substantiated that the NVs presenting PD-1 on the surface can effectively interact with tumor cells through the binding between PD-1 receptor and PD-L1.

To investigate the systemic biodistribution and kinetics of PD-1 NVs, the free NVs and PD-1 NVs were labeled with Cy5.5. Free NVs and PD-1 NVs were injected into the mice through tail-vein. As shown in FIG. 3f, the PD-1 NVs had higher blood retention compared to the free NVs. The PD-1 NVs exhibited 29% and 13% overall retention compared to 12% and 1.7% retention of the free NVs at 8 h and 24 h, respectively. Next, the in vivo tissue distribution of PD-1 NVs was examined. B16F10-tumor-bearing mice received Cy5.5 labeled PD-1 NVs via tail vein injection. Notably, the accumulation of Cy5.5 fluorescence of PD-1 NVs was observed primarily at the liver, kidney and tumor sites (FIGS. 3g and 3h). To further assess the biodistribution of the PD-1 NVs, the Cy5.5 labeled NVs were quantified in the sections of organs and tumors by confocal imaging. The WGA Alexa-Fluor 488 dye was used to stain the cell membrane in the tissue sections. The distribution of the PD-1 NVs paralleled the imaging data showing intensive accumulation of the PD-1 NVs in tumor tissue sections (FIG. 3i).

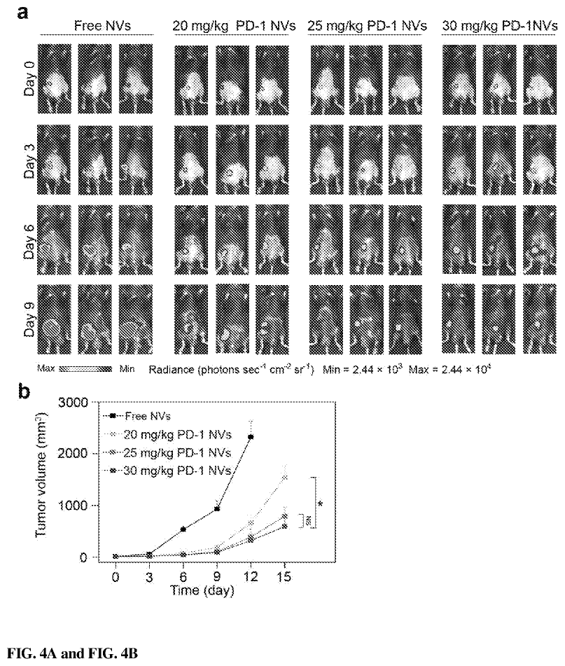

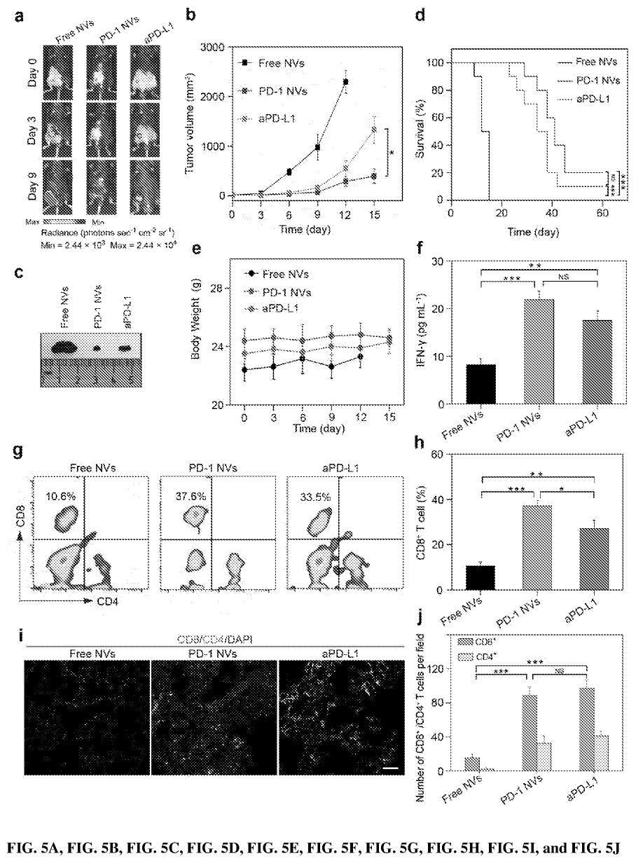

To determine whether the PD-1 NVs promote the mice immune response to the melanoma tumor, a melanoma tumor model was established in which B16F10-luc cells were inoculated subcutaneously in C57BL/6 mice. Five days after tumor inoculation, 25 mg/kg free NVs and 20-30 mg/kg PD-1 NVs were inoculated in mice through tail-vein injection. Tumor growth was monitored by measuring both bioluminescence signals and tumor size. Notably, the growth of B16F10 tumors was significantly delayed in mice treated with PD-1 NVs at the dosage of 20 mg/kg, 25 mg/kg and 30 mg/kg (FIG. 4). PD-L1 antibody is a clinical therapeutic antibody to block PD-L1 for melanoma treatment. To confirm the in vivo anti-tumor effect of PD-1 NVs, treatment with the administration of the anti-PD-L1 antibody as a positive control was employed. The mice were divided into three group: 25 mg/kg free NVs (Group 1) and PD-1 NVs (Group 2) were injected in mice through tail-vein injection every three days for five cycles. Anti-PD-L1 antibody (aPD-L1, Group 3) was also injected into mice at 2 mg/kg as a positive control group. Tumor growth was monitored using both bioluminescence signals and tumor size. Of note, PD-1 NVs significantly delay the B16F10 melanoma tumor growth, comparable to the treatment with aPD-L1. (FIGS. 5a, 5b, and 5c). Consequently, PD-1 NVs improved the survival of the mice (FIG. 5d), and 20% of mice survived more than 60 days upon PD-1 NVs treatment. Moreover, there was no obvious weight loss during the treatment (FIG. 5e). No significant anti-tumor effects were observed in mice treated with free NVs.

Exhausted CD8⁺ T cells express inhibitory receptor proteins, including PD-1, TIGIT, LAG3 and TIM3, and have reduced capacity to produce immune cytokines, such as IFN-γ and TNF-α. To assess whether PD-1 NVs treatment reduce T cell exhaustion and maintain their anti-tumor function, IFN-γ and TNF-α levels were measured in the serum of the treated mice by the end of the fifth cycles. IFN-γ levels in the serum of mice treated with either PD-1 NVs or aPD-L1 were significantly increased (FIG. 5f), while TNF-α levels remained unchanged. The infiltration of CD8⁺ T cells in the harvested tumor was analyzed by flow cytometry. The percentage and numbers of activated CD8⁺ T cells were significantly increased in tumor collected from mice treated with either PD-1 NVs or aPD-L1 groups as compared to control group (FIGS. 5g and 5h). Similarly, higher densities of CD8⁺ T cells were detected by immunofluorescence in tumors collected from mice treated with either PD-1 NVs or aPD-L1 (FIGS. 5i and 5j). Finally, the potential toxicities caused by PD-1 NVs was also evaluated. After five cycles of treatments, blood cell counts (CBC) showed that lymphocytes and monocyte content slightly decreased in mice treated with PD-1 NVs, while the lymphocyte ratios were not affected. Additionally, the plasma level of Immunoglobulin E (IgE) antibody, produced by the immune system overreacts to an allergen, did not significantly increase after five cycles of the treatment with PD-1 NVs.

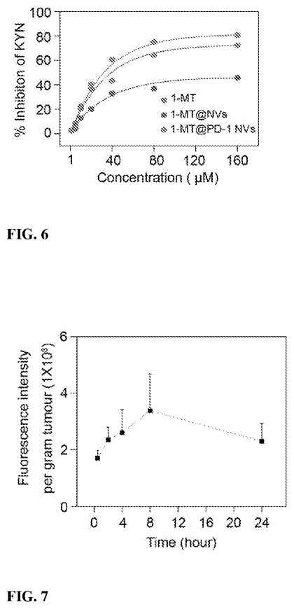

Next, the IDO inhibitor 1-MT was loaded into the PD-1 NVs to investigate the combinatorial therapy of IDO inhibitor and immune checkpoint blockage. High loading efficiency (24.5%) of 1-MT was achieved by employing the electric shock method compared to the traditional incubation methods (16.5%). The release of 1-MT from the PD-1 NVs was also tested. 1-MT can be rapidly released from the NVs within 24 hours in vitro. Furthermore, to determine the inhibitory effect of 1-MT released by 1-MT-loaded PD-1 NVs, an IDO inhibition assay was performed using HeLa cells that express IDO after IFN-γ stimulation. Remarkably, PD-1 loaded 1-MT had better inhibitory effect compared to the free 1-MT and 1-MT loaded free NVs (FIG. 6). To evaluate the in vivo drug release in the tumors, the accumulation of PD-1 NVs in the tumor was detected. Cy5.5 labeled PD-1 NVs were accumulated in the tumors within 30 min post injection and the accumulation gradually increased over time, which indicated that 1-MT can be effectively released in the tumors (FIG. 7).

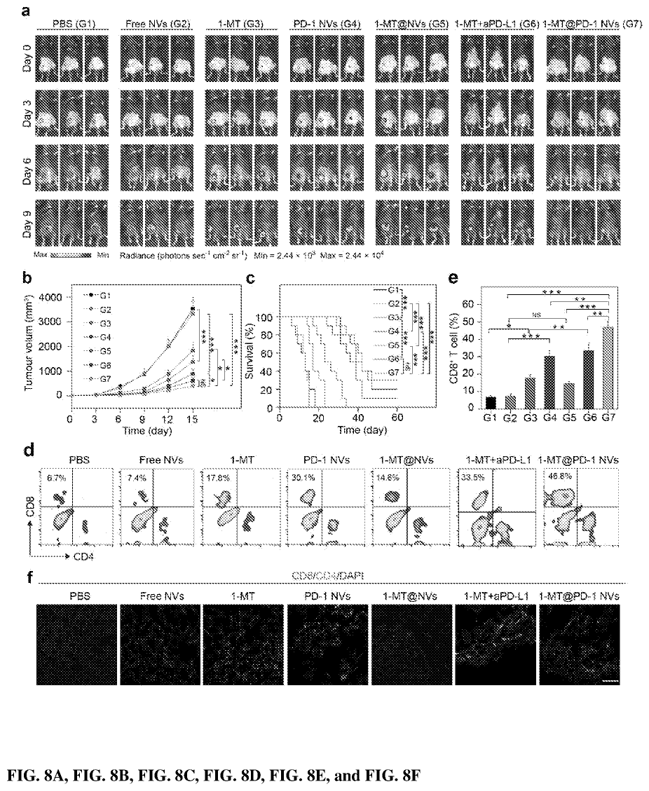

To demonstrate that the simultaneous IDO inhibition and PD-L1 blockade provided by 1-MT-loaded PD-1 NVs enhances anti-tumor activity, B16F10-luc tumor bearing mice were treated with either PBS (Group 1), free NVs (Group 2), free 1-MT (Group 3), PD-1 NVs (Group 4), 1-MT loaded free NVs (Group 5), 1-MT plus aPD-L1 (Group 6) or 1-MT loaded PD-1 NVs (Group 7) every 3 days for five cycles. Tumor growth were monitored by measuring both bioluminescence signals and sizes of the tumors. A high response rate (>80%) in mice treated with free 1-MT and 1-MT loaded free NVs (60%) was found, however, limited suppression of tumor growth was observed (FIGS. 8a and 8b). This non-ideal efficacy may be because multiple immune suppression mechanisms exist within the TME. Notably, PD-1 NVs had better anti-tumor effects as compared to 1-MT (FIGS. 8a and 8b). Mice treated with 1-MT plus aPD-L1 exhibit significantly delayed the progress of the melanoma tumors (FIGS. 8a and 8b). Importantly, treatment with 1-MT loaded PD-1 NVs showed>80% responses to the melanoma tumor, which is much more efficiently than the treatment with 1-MT or PD-1 NVs alone (FIGS. 8a and 8b), and are comparable to the treatment with 1-MT plus aPD-L1 (FIGS. 8a and 8b). Furthermore, the dual inhibition of IDO and PD-L1 by 1-MT loaded PD-1 NVs improved the survival of the treated mice without obvious weight loss (FIG. 8c). The density of the CD8⁺ T cells was examined in the tumor margin of different treatment groups. Tumor infiltrated CD8⁺ T cells from tumor in all the treatment groups were harvested and analyzed by flow cytometry and immunofluorescence. It was demonstrated that treatments with free 1-MT and 1-MT loaded NVs increased the number of infiltrating CD8⁺ T cells by approximately 15-20% compared to the PBS-treated group (FIGS. 8d and 8e). Immunofluorescence staining confirmed that PD-1 and 1-MT loaded PD-1 NVs significantly enhanced the density of tumor-infiltrated CD8⁺ T (FIG. 8f). The therapeutic efficacy of combination treatment was better than the individual ones. Infiltration of CD4+ FoxP3⁺ T cells was also studied. Notably, CD4+ FoxP3+ T cells were reduced in 1-MT loaded PD-1 NVs group as well compared to control group. Finally, major organs such as liver, spleen, kidney, heart and lung were collected and assessed by immunohistochemistry without showing any obvious sign of organ damage. These data revealed that IDO inhibition combined with PD-L1 blockage PD-1 NVs significantly disrupted the immunosuppression of TME, which enhanced the elimination of cancer cells by the host's immune system. 82. In summary, cellular nanocarriers displaying PD-1 receptors were engineered that effectively bind to PD-L1 on the tumor cells and disrupt the PD-1/PD-L1 inhibitory axis. PD-L1 blockade by PD-1 NVs significantly enhanced the immune response against the melanoma tumor in vivo. Furthermore, PD-1 NVs can also be adapted to carry a variety of therapeutics to achieve a synergistic efficacy. IDO inhibition and PD-L1 blockade were achieved by 1-MT-loaded PD-1 NVs. The simultaneous disruption of dual immune tolerance mechanisms in tumors remarkably suppressed the melanoma tumor growth in vivo. Thus, PD-L1 blockade by PD-1 cellular NVs provides a promising strategy that leverages functions of both delivery vehicles and encapsulated drugs for enhancing immunotherapy.

### a) Methods and Materials

### (1) Chemical and Regents

1-MT, Hygromycin B, phosphatase inhibitor cocktail, Optiprep solution were ordered from Sigma-Aldrich. mGM-SF, mIL-4 and mTNF-α were ordered from Thermo Fisher Scientific. PD-L1 antibody was from Thermo Scientific. Anti-PD-1 antibody for western blot was from Sigma-Aldrich. Mouse CD4 and CD8 antibodies for immunofluorescence staining were ordered from Abcam. Anti-PD-L1 antibody (aPD-L1) used in vivo was purchased from Biolegend Inc. Protein A/G-agarose beads were purchased from Santa Cruz. Wheat Germ Agglutinin (WGA) Alexa Fluor 488 and 594 dyes were purchased from Thermo Scientific. Staining antibodies included CD3, CD4 and CD8, for FACS analysis were order from Biolegend Inc.

### (2) Plasmid and Cell Line

Mouse PD-1 was cloned into pCMV6 mammalian expression vector. Plasmids was confirmed by automated DNA sequencing. Mouse EGFP-PD-L1 plasmid was purchased from Sino Biological. HEK293T cells were transiently transfected with the plasmids using lipofectamine 2000 (Invitrogen) according to the manufacturer's instructions. To establish stable cells, HEK293T cells were transfected with pCMV6-OFR-PD-1 and further selected with hygromycin B. B16F10 cells were transfected with EGFP-PD-L1 plasmid using Lipofectamine™ Transfection Reagent (Invitrogen, 18324012).

### (3) Cell Culture

HEK293T cells were cultured in Dulbecco's Modified Eagle's Medium (DMEM) supplemented with 10% Fetal Bovine Serum (FBS). The mouse melanoma cell line B16F10 was purchased from the American Type Culture Collection. For in vivo bioluminescent tumor imaging, B16F10-luc cells were gifts from Dr. Leaf Huang at UNC. HeLa cells were obtained from Tissue Culture Facility of UNC Lineberger Comprehensive Cancer Center. Cells isolated from bone marrow of C57BL/6 mice were cultured in RPMI 1640 with 10% FBS complement with 20 ng mGM-CSF and 10 ng IL-4 to obtained bone marrow-derived DCs.

### (4) Prepare Cell Membrane Nanovesicles

HEK293T cells stably expressing DsRed-PD-1 were cultured in DMEM medium with 10% FBS. The cells were harvested with trypsin. The cells were washed with cold PBS for 3 times by centrifuging at 1000 rpm. Then, the cells were suspended with homogenization medium (HM) containing 0.25 M sucrose, 1 mM EDTA, 20 mM Hepes-NaOH, pH 7.4, and protease inhibitor cocktail. After that, the cells were disrupted by using a dounce homogenizer for at least 50 times on ice. The entire solution was spun down at 1000×g for 5 min. And then, the pellet was discarded and supernatant centrifuged at 100,000×g for 1 h.

The pellet containing plasma membrane material was washed with HM buffer for 3 times. To prepare cell membrane nanovesicles, the cell membranes in HM buffers were passed through 0.8 μm filters for at least 10 times, and then passed through 0.22 μm filters for another 10 times. To further purify the nanovesicles, the extruded samples was subject to a step gradient of 50% iodixanol (Optiprep) and then ultracentrifuged at 100,000×g for 2 h at 4° C.

### (5) Isolation of DC Cells from Mouse Bone Marrow

Bone marrow-derived DCs were isolated from bone marrow. In brief, the femurs and tibias were isolated from C57BL/6 mice and keep in RPMI 1640 medium on ice. The end of each bone was cut off with scissors, and the marrow was flushed with 2 mL RPMI 1640 medium with a syringe. The medium containing cells were passed through Nytex mesh to remove the large particles. Centrifuge the cells at 1000 rpm for 5 min, discard the supernatant. The cells were suspended with red cell lysis buffer (Thermo Scientific) to lysis red cells for 5 min at room temperature. Wash the marrow cells twice with RPMI 1640, each time by centrifuging 10 min at 1000 rpm at room temperature. Seeded the cells in the culture dish with RPMI 1640 medium and supplemented with mouse granulocyte/macrophage colony-stimulating factor (mGM-CSF, 50 ng/mL) and IL-4 (10 ng/mL). The aggregates of the cells can be observed between day 5 and day 8. Dislodge aggregates was gently dispersed by RPMI 1640 medium and seeded the cells in a 6 well plate with RPMI 1640 supplemented mGM-CSF, 50 ng/mL and IL-4 for further use.

### (6) 1-Mt Loading

To load 1-MT to PD-1 NVs, 1 mg purified vesicles and 500 μg 1-MT (100 mg/mL diluted in PBS at pH10) were gently mixed in 1 ml electroporation buffer (1.15 mM potassium phosphate pH 7.2, 25 mM potassium chloride, 21% Optiprep) at 4° C. The samples were subjected to electroporation at 300 V and 150 μF in 0.4 cm electroporation cuvettes using a MicroPulser Electro-porator (Bio-Rad, USA). After that, the electroporation cuvettes containing samples were incubated on ice for 30 min for the membrane recovery. NVs were then washed with cold PBS by ultracentrifugation at 100,000×g for 3 times. For the other method to load 1-MT to nanovesicles, 1 mg purified vesicles and 500 μg of 1-MT, were gently mixed in 1 ml PBS and incubate for 2 h at 37° C. Nanovesicles were then washed with PBS for three times.

### (7) Western Blot

Immunoblotting analysis was performed. For abbreviation, HEK293T cells with stably expressing DsRed-PD1 were lysed with RIPA lysis buffer (Thermo Scientific). And then, cell lysates and purified membrane vesicles samples were resolved on 12% SDS-PAGE and analyzed by immunoblotting using PD-1 and R-actin antibodies, followed by enhanced chemiluminescence (ECL) detection (Thermo Scientific).

### (8) Co-Ip Assay

To detect the interaction between PD-1 on nanovesicles (NVs) and PD-L1 on B16F10 cells, the Co-IP assays were carried out. Briefly, 1 mL (700 μg/mL) PD-1 NVs were added and incubated with B16F10 cells (10 cm dish) for 20 h. After that the cells were washed with PBS for three times to remove the un-binding NVs. And then, the cells were lysed in 1 ml RIPA lysis buffer (Thermo Scientific) containing phosphatase inhibitor cocktail. The cell lysis was clarified by centrifugation at 15000×g for 10 min at 4° C. Clarified lysates were pre-cleared with protein G-agarose beads (Santa Cruz) for 1 h at 4° C. with gentle rotation. Then the cell lysis was incubated with PD-1 primary antibody shaking overnight at 4° C. The next day with 10 μL of protein G-agarose beads for 2 h at 4° C. The beads were washed gently with ice-cold RIPA buffer 5 times. The bound proteins were resolved by 12% SDS-PAGE and analyzed by immunoblotting using the indicated antibodies.

### (9) Immunoprecipitation Assay (IP Assay)

To detect the orientation of PD-1 receptors, the immunoprecipitation (IP) assay was performed. Briefly, 1 mL (500 μg/mL) of PD-1 NVs were pre-incubated with protein A/G beads for 1 h at room temperature to remove the nonspecific binding proteins. Then, the PD-1 NVs were incubated with 2 μg PD-1 primary antibody for 5 h at 4° C. After that 10 μL of protein A/G-agarose beads was added and incubate for 2 h at room temperature. The beads were washed gently with PBS for 3 times. The bound proteins were resolved by 12% SDS-PAGE and analyzed by immunoblotting using the indicated antibodies.

### (10) Nanovesicles Cell Binding Assay

B16F10 cells were seeded in confocal dishes. DsRed-PD-1 NVs (50 μg/mL) or PD-1 free NVs labeled with Cy5.5 (50 μg/mL) were added to the medium and incubate for 20 h. aPD-L1 antibody (20 μg/mL) were incubated with the cells for 4 h before the PD-1 NVs were added in the culture medium as indicated. After that Wheat Germ Agglutinin (WGA), Alexa Fluor 488 conjugate was added to label the cell membranes for 10 min. The BMDCs (7 days after isolation from bone marrow) were seeded in confocal wells, and 10U TNF-α was add to stimulate the DC cells for maturation. Then, DsRed-PD-1 NVs (50 μg/mL) were added and incubated with DC cells for other 20 h. After that Wheat Germ Agglutinin (WGA), Alexa Fluor 488 conjugates were added to label the cell membranes for 10 min. Then, nuclei were stained with Hoechst for 10 min. The cells were washed with PBS for three times. Confocal microscopy was performed on confocal microscope (Zeiss) in sequential scanning mode using a 63×objective. For flow cytometric analysis of PD-1 NVs binding with B16F10 cells PD-1 NVs (50 μg/mL) were incubated with B16F10 cells for 2 h. Or aPD-L1 antibody (20 μg/mL) were incubated with the cells for 4 h before the PD-1 NVs were added in the culture medium as indicated. Gated on DsRed⁺.

### (11) Drug Release

The 1-MT release of NVs (1 mg/mL) was analyzed in PBS (pH7.2) at 37° C. The amount of released 1-MT was detected by HPLC. The separation was performed in sodium acetate buffer (50 mM, pH 4.2) with an increasing gradient of acetonitrile, using a flow rate of 1.0 mL/min. The absorbance of the column effluent was monitored at 280 nm.

### (12) Cellular Assay for IDO Activity

To detect the inhibit effect of 1-MT on IDO, for IDO enzyme activity assays, HeLa human tumor cells were seeded at 4.0×10⁴ cells well in DMEM/phenol red free media supplemented with 80 μM L-tryptophan, 10% FBS (Hyclone) and penicillinstreptomycin (Gibco). The following day, 1-MT or the 1-MT@PD-1 NVs were solubilized in DMSO/0.1 N HCl and serially diluted in assay wells while maintaining the DMSO/HCl dilution constant at 1:1000. The 100 ng/mL of human recombinant IFN-γ (cat. #570206, BioLegend Inc. San Diego, Calif.) was then added per well to stimulate IDO expression. Tryptophan was qualified by a fluorescence detector at an excitation wavelength of 285 nm and an emission wavelength of 365 nm or by HPLC at 280 nm.

### (13) Circulation

Free NVs and PD-1 NVs were labeled by NHS-Cy5.5 in PBS buffer. Following incubation overnight at 4° C., Cy5.5-labeled PD-1 NVs were washed with PBS for 3 times. The C57BL/6 mice were injected with 200 μL (2 mg/mL) Cy5.5 labeled Free NVs and PD-1 NVs through tail-vein, respectively. The blood of the mice was collected from the eye socket at different time points (at 2 min, 2 h, 4 h, 8 h, 24 h and 48 h, respectively) post-injection. Then the fluorescence signal of serum was measured.

### (14) Biodistribution

PD-1 NVs were labeled with NHS-Cy5.5 in PBS buffer. Following incubation overnight at 4° C., Cy5.5-labeled PD-1 NVs were washed with PBS for three times. The melanoma tumor bearing C57BL/6 mice were injected with 200 μL (2 mg/mL) Cy5.5 labeled PD-1 NVs through tail-vein. The control group was injected with PBS. After 24 h and 48 h, major organs and tumors of mice were harvested. Finally, fluorescence imaging and average fluorescence intensities were recorded using a Xenogen IVIS Spectrum imaging system.

### (15) In Vivo Anti-Tumor Efficacy Study

Female C57BL/6 mice were purchased from Jackson Lab (USA). All mouse studies were performed in the context of an animal protocol approved by the Institutional Animal Care and Use Committee at North Carolina State University and University of North Carolina at Chapel Hill. Mice were weighed and randomly divided into different groups. 5 d after 1×10⁶ B16F10 tumor cells subcutaneously transplanted into the abdomen of mice (the tumor reaches 40-50 mm³), PBS, Free nanovesicles (25 mg/kg), PD-1 nanovesicles (25 mg/kg), 1-MT (2.5 mg/kg), 1-MT loaded PD-1 nanovesicles (25 mg/kg), anyi-PD-L1 antibody (2 mg/kg) were administered into mice by tail-vein injection. Tumor incidences were monitored by physical examination and sizes were also measured by digital caliper over time. Tumors were measured by using a vernier calipers and the volume (V) was calculated to be V=d²×D/2, where d is the shortest and the D is longest diameter of the tumor in mm respectively. To assess potential toxicities, mice were monitored daily for weight loss. For survival assays, the experiments were performed separately.

### (16) In Vivo Bioluminescence and Imaging

Bioluminescence images were collected with a Xenogen IVIS Spectrum Imaging System. Living Image software (Xenogen) was used to acquire the data 10 min after intraperitoneal injection of d-luciferin (Pierce) in DPBS (15 mg/mL) into animals (10 μL/g of body weight).

### (17) Tissue Immunofluorescence Assay

Tumors were dissected from the mice and snap frozen in optimal cutting medium (O.C.T.). Several micrometer sections were cut using a cryotome and mounted on slides. The frozen organs (lung, liver, heart, kidney, spleen) and tumor sections were incubated in PBS for 15 min to remove the embedding medium. The specimens were blocked with the buffer containing 3% BSA and 0.5% Triton X-100 for 30 min. For the organs, the specimens were incubated with WGA Alexa Fluor 488 for 10 min. For the tumor specimens, tumor sections were subsequently, incubated with CD4 and CD8 primary antibodies (1:50 in 1.5% BSA) overnight and then washed three times with PBS for 5 min each. They were then incubated with TRITC secondary antibody (KPL) diluted in 1.5% BSA at room temperature in the dark for 1 h. Finally, the nucleus was stained with DAPI, and the tissue was washed three times with PBS for 5 min each. Confocal microscopy was performed on a FLUO-VIEW laser scanning confocal microscope (Zeiss) in sequential scanning mode using a 40×objective.

### (18) Cytokine Detection

Plasma samples were isolated from mice after various treatments and diluted for analysis. Tumor necrosis factor (TNF-α, Invitrogen), interferon gamma (IFN-γ, eBioscience), were analyzed with ELISA kits according to manufacture' protocols.

### (19) H&E Staining

The major organs (liver, spleen, kidney, heart and lung) of the mice received different treatments were harvested and fixed in 10% neutral buffered formalin. Then the organs processed routinely into paraffin, sectioned at 8 μm, stained with haematoxylin and eosin, and finally examined by digital microscopy.

### (20) Statistical Analysis

All results are expressed as the mean±s.d. or the mean±s.e.m. as indicated. Biological replicates were used in all experiments unless otherwise stated. One-way or two-way analysis of variance (ANOVA) and Tukey post-hoc tests were used when more than two groups were compared (multiple comparisons) as indicated. Survival benefit was determined using a Log-Rank test. All statistical analyses were performed using the IBM SPSS statistics 19. The threshold for statistical significance was P<0.05.

### 2. Example 2: Platelets Expressing PD-1 for Cancer Immunotherapy

Currently, there are many intrinsic and extrinsic mechanisms of resistance to immunotherapy beside of PD-L1, including loss of tumor antigen expression, CTLA-4 and other immune checkpoints, and immune suppressive cell populations (Tregs, MDSC, type II macrophages). Among these immune blockades, CD4⁺ CD25⁺ FoxP3⁺ regulatory T cells (T_{reg} cells) compete in the consumption of IL-2 in the tumor microenvironment, which suppress the proliferation of tumor infiltrated CD8⁺ T cells. Moreover, activated T_{reg} cells can also directly kill T cells through perforin. Thus, abundant T_{reg} cells in tumor tissue is a crucial obstacle of successful cancer immunotherapy. Depletion of T_{reg} cells significantly improve the response rate of PD-1/PD-L1 blockade.

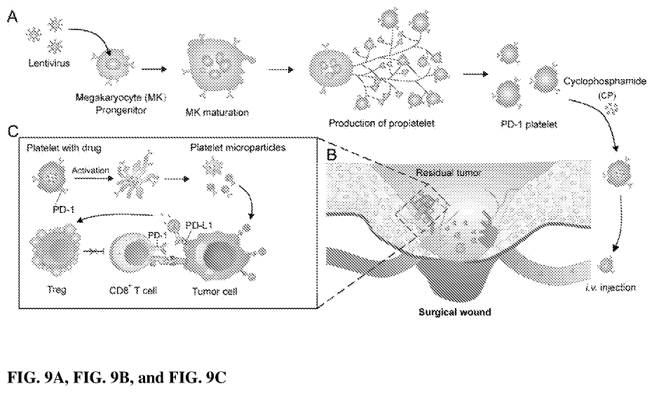

As the monitor of vascular damage, invasive microorganisms and circulating tumor cells (CTCs) in bloodstream, platelets have been recently used to design nanocarriers. Platelets conjugated with anti-PD-L1 can target the tumor surgery wounds to reinvigorate the exhausted CD8⁺ T cells and thus reduce post-surgical tumor recurrence and metastasis. However, blood-originated platelets present a biosafety and insufficiency challenge due to need of a large amount of host-matched platelets during the treatment. In addition, platelets are anucleate, which cannot proliferate or be genetically manipulated. Alternatively, in vitro production from Megakaryocytes (MKs) can provide large-scale source of platelets. Herein, megakaryocytes were genetically engineered for stable expression of mouse PD-1 and subsequently produced platelets presenting PD-1 in vitro. These cells were then applied to the surgical wound via reinvigoration of exhausted CD8⁺ T cells (FIGS. 9A, 6B, and 9C). In addition to PD-L1 blockade, PD-1-expressing platelets can also carry and transport cyclophosphamide, which allows the depletion of Tregs within the tumor microenvironment and further enhance the antitumor effects of CD8⁺ T lymphocyte cells within the surgical tumor microenvironment.

Besides blockade PD-L1, PD-1 platelets also can function as a platform and combine with other immune blockade inhibitors to improve the response rate. Therefore, cyclophosphamide was simultaneously loaded into the platelets to deplete Treg cells. Cyclophosphamide loaded PD-1 platelets formulation disrupted the immune blockade of PD-L1 and Treg cells, which significantly increased the frequency of reinvigorated CD8⁺Ki67⁺GrzmB⁺ lymphocyte cells in surgical tumor microenvironment. Thus, PD-1 platelets as a cell platform combined with other immune blockade inhibitors can improve the response rate and reduce the rate of tumor relapse after surgery.

### (1) Generation of MKs Cell Lines Stable Expressing PD-1

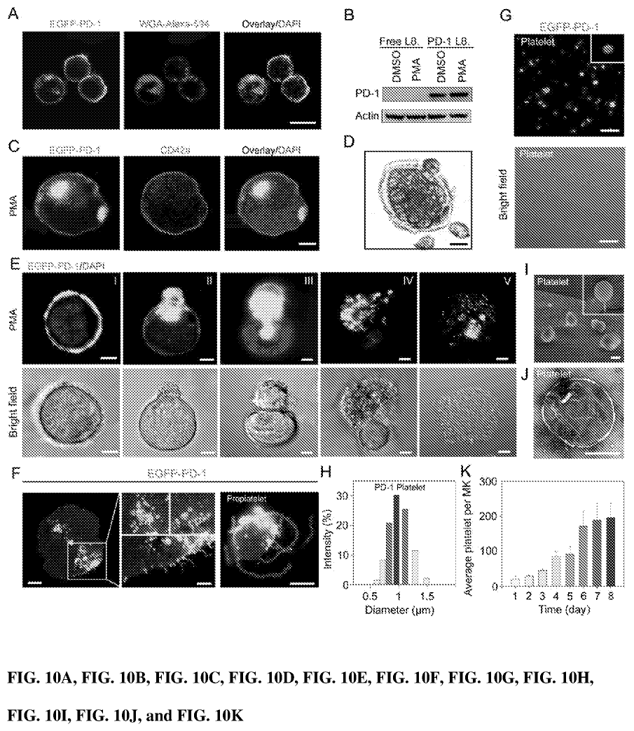

Platelets are released from the bone marrow and lung resident MKs. To produce the platelets in a large-scale, the murine MKs progenitor cell L8057 were treated with phorbol 12-myristate 13-acetate (PMA). After the stimulation, the cell volume was significantly increased and accompanied with the proplatelet extension and platelet release. MKs with larger cell volume contained multiple nuclear, indicating the maturation and ready for releasing the platelets. To generate PD-1 platelets, L8057 cell lines stably expressing mouse EGFP-PD-1 was established by transducing with lenti-virus and post-screened with puromycin. Remarkably, PD-1 receptors were expressed and localized on the cell membranes, indicated by the co-localization of fluorescence from EGFP and the cell membrane dye Alexa Fluor 594 conjugate wheat germ agglutinin (WGA594) (FIG. 10A). PD-1 expression on EGFP-PD-1 L8057 cells was confirmed by western blot (FIG. 10B). CD41a, the marker of MKs, was intensively expressed on PD-1 L8057 cell line. After the stimulation with PMA, PD-1-expressing L8057 cells underwent maturation, and morphologically displayed typical peripheral nuclei and increased cytoplasmic volume. CD42a, a marker of MK maturation, was expressed on the cell membrane (FIG. 10C). Moreover, the platelet surface receptors GPVI (collagen receptors) and P-Selectin were expressed in mature PD-1 L8057 cells. Wright-Giemsa staining revealed that mature PD-1 L8057 cells contained polyploid nuclei (FIG. 10D).

### (2) In Vitro Production of PD-1 Platelets from MKs

Mature MKs typically reside in bone marrow and lung budding podosomes and prolong to form proplatelets. Proplatelets cross through the sinusoidal endothelium and release platelets into the bloodstream. Similarly, mature PD-1-expressing L8057 cells had budding podosomes, which prolonged to form the proplatelets (FIG. 10E). Notably, the proplatelets were budded and extended from the cell membranes to form pearl-like structures (FIG. 10F). The proplatelets finally disbanded and released platelets. MK cytoplasm containing EGFP-PD-1⁺ membrane vesicles existed as a membrane reservoir for proplatelet formation (FIG. 10F). These PD-1-expressing membrane vesicles fused to form tubular structure and budded from the cell surface (FIG. 10F). Purified platelets from the culture media showed green fluorescence indicating that PD-1 was present in the platelets (FIG. 10G). Binding receptors including GPVI and P-Selectin were also expressed in platelets released from L8057 cells. Moreover, DLS analysis showed that the average diameter of the platelets was around 2 μm and with a zeta potential of −10±2.6 mV (FIG. 10H). As documented by cryo-scanning electron microscopy (CSEM) and transmission electron microscopy (TEM), purified platelets showed spherical morphology (FIGS. 10I and 10J). Further the platelet production from PD-1 L8057 cells was quantitatively measured, the production of platelets significantly increased at day 6 after the stimulation with PMA (FIG. 10K).

### (3) Biological Behavior of PD-1 Platelets

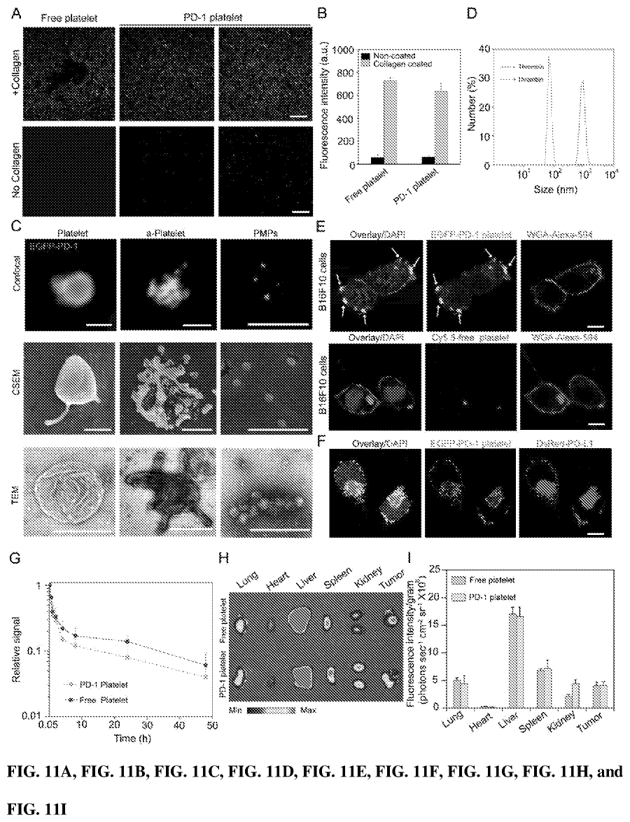

Platelets can achieve hemostasis, recruit other leukocytes for host defense responses, and release several immunoactive molecules. Platelet activation occurs after adhering to vascular lesions. Collagen is the primary sub-endothelial component for active platelets binding. Therefore, collagen binding property of PD-1 platelets were tested. Indeed, WGA Alexa-Fluor 594 dye labeled free and PD-1 platelets had strong collagen adhesion ability (FIGS. 11a and 11b). In contrast, blockade of the collagen receptor GPVI with anti-GPVI antibodies, intensively reducing the collagen adhesion ability of the platelets. Thrombus formation by platelets aggregation is another critical event for haemostatic response. In response to agonist stimulation with thrombin, free and PD-1 platelets efficiently aggregated between themselves in response to agonistic stimulation with thrombin. In addition, platelet microparticles (PMPs) are generated from activated platelets carrying chemokines and adhesion molecules, facilitating monocyte in inflammation and atherosclerosis site. To examine whether PMPs can be generated from activated PD-1 platelets on stimulation, the platelets were treated with thrombin in vitro. CLSM, SEM, and TEM images indicated the generation of PMPs from activated platelets (FIG. 11c). It was also observed that the platelet morphology became more dendritic and expansive after the treatment with thrombin (FIG. 11c). Furthermore, DLS analysis detected the generation of smaller particles, indicating the PMPs released from activated platelets (FIG. 11d).

Elevation of PD-L1 expression on tumor cells turned T cells exhausted (Tₑₓ). To investigate whether PD-1 platelets could bind to the surface of the melanoma cancer cells and blockade PD-L1, the PD-1 platelets were incubated with B16F10 melanoma cancer cells in vitro. Of note, PD-1 platelets effectively bound to B16F10 cells and were then internalized by the cancer cells (FIG. 1e). In contrast, the free platelets showed limited ability to bind to the B16F10 cells (FIG. 1e). To examine whether the PD-L1/PD-1 interaction mediates the internalization of platelets, anti-PD-L1 antibody was added to block PD-L1 on the B16F10 cells. The confocal images showed that PD-1 platelets binding was significantly reduced when PD-L1 antibody was pre-incubated with the cells. Furthermore, the EGFP-PD-1 platelets colocalized with PD-L1 ligands on B16F10 melanoma cells, indicating the interaction between PD-1 and PD-L1 (FIG. 11f). To investigate the systematic in vivo circulation time of free and PD-1 platelets, platelets were labeled with Cy5.5 and were subsequently injected into the mice through tail-vein injection. Free platelets had a bit longer (14%, 24 h) blood retention property compared to the PD-1 platelets (8%, 24 h) (FIG. 11g). When Cy5.5-labeled platelets were inoculated intravenously after tumor resection in B16F10 tumor-bearing mice, both free and PD-1 platelets could be accumulated in the residual tumor bed (FIGS. 11H and 11I). Meanwhile the platelets intensively accumulated in the liver and spleen (FIGS. 11H and 11I). Glycoprotein VI (GPVI) is the collagen receptor on the platelets and responsible for the platelets to target the wound. PD-1 platelets and free platelets showed similar binding ability on the collagen (FIG. 11A). Therefore, the accumulation ability in the surgical tumors is similar between the free platelets and PD-1 platelets (FIG. 11H).

### (4) In Vivo Anti-Tumor Effect of PD-1 Platelets

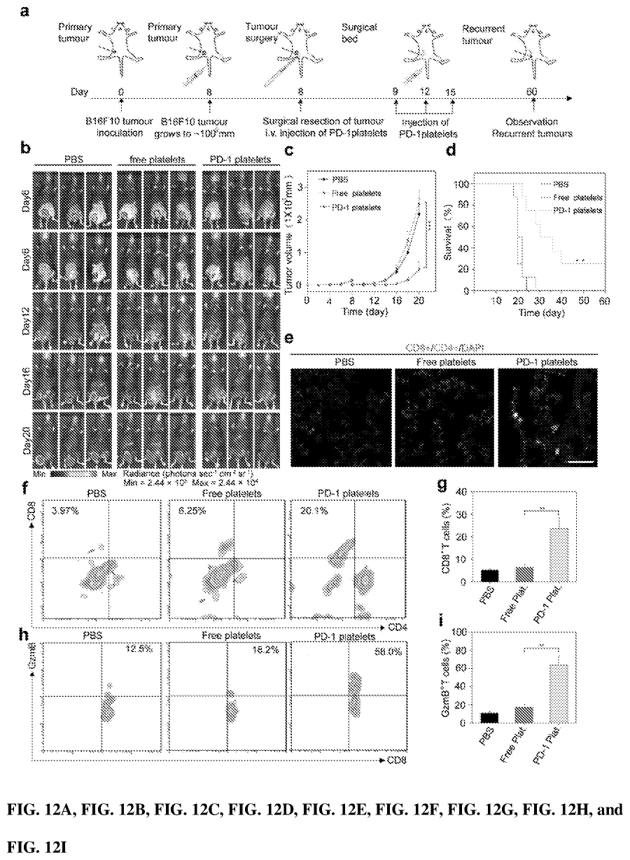

Upregulation of PD-L1 on melanoma cells turns T cells exhausted, exhibiting T cells dysfunction in proliferation and activity. To investigate whether PD-1 platelets could blockade PD-L1 to regress the residual tumor after surgery, the B16F10 melanoma incomplete-tumor-resection model was used to mimic post-surgical local relapse (FIG. 12a). When the tumor volume growth around 100 mm³, the mice were intravenously injected with a single dose of phosphate-buffered saline (PBS), free platelets (1×10⁸), PD-1 platelets (1×10⁸). After the mice receiving the platelets injection, tumor surgery was immediately carried out to remove most of the tumor (˜90%). After the surgery, the mice received additional treatment during the period of wound healing (FIG. 12a). Notably, high response rate was achieved in the mice that received PD-1 platelets as assessed by monitoring the tumor bioluminescence and measuring the tumor size. (FIGS. 12b and 12c). The progress of the residual tumor was significantly delayed in the mice that received PD-1 platelets by monitoring the bioluminescence signal of B16F10 cells and the measurement of the tumor size (FIGS. 12b and 12c). In contrast, residual melanoma tumors were rapidly progressed in the mice that received free platelets or PBS (FIGS. 12b and 12c). Benefiting from the PD-1 platelets treatment, 25% of mice survived more than 60 days without obvious weight loss or other signs of toxicities (FIG. 12d). There was no obvious sign of organ damage were observed in the platelets treated mice. To exam the accumulation of CD8⁺ TILs, the tumors were collected and analyzed by fluorescence-activated cell sorting (FACS) and immunofluorescence. Remarkably, the frequency of CD8⁺ TILs intensively increased in the tumor of PD-1 platelets treated mice (FIGS. 12E, 12F, and 12G, and T cells exhibited increased expression of cytotoxic protein granzyme B (GzmB), indicating that PD-1-expressing platelets can revert T cell exhaustion within the tumor microenvironment (FIGS. 12H and 12I)

### (5) In Vivo Anti-Tumor Effect of Cyclophosphamide Loaded PD-1 Platelets

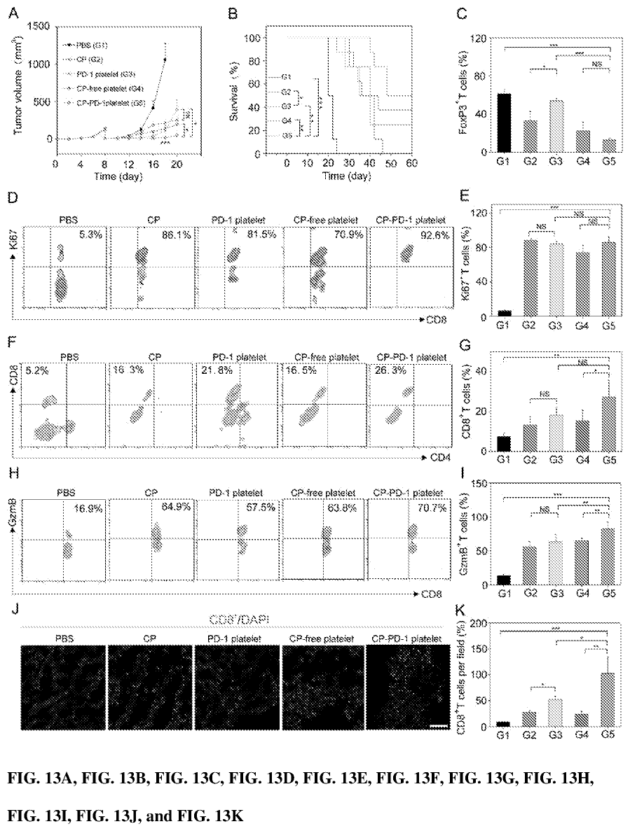

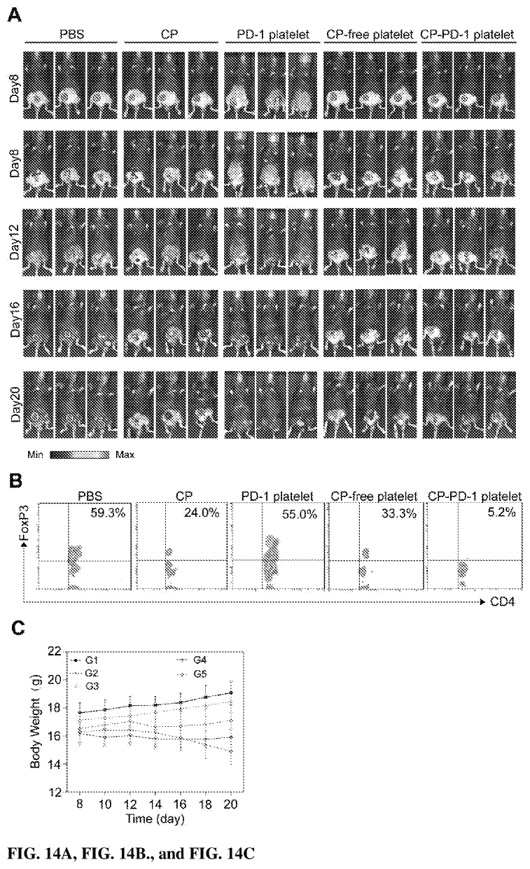

Low doses of cyclophosphamides can improve immune responses in various murine tumor models and patients, which is generally attributed to selective depletion of Tregs. To counter Tregs at the tumor site, we loaded the cyclophosphamide into the platelets. It was found that platelets could internalize and release cyclophosphamide within 24 h in vitro. To investigate the simultaneous anti-tumor effect of PD-L1 blockade and cyclophosphamide-induced depletion of Tregs, the same B16F10 melanoma model with incomplete-tumor-resection was used. In this model, while cyclophosphamide and PD-1-expressing platelets showed limited results when used as single agents (FIG. 13A and FIG. 14A), tumor progression was significantly suppressed in mice treated with cyclophosphamide-loaded PD-1-expressing platelets (P<0.001) (FIG. 13A and FIG. 14A). Treg depletion by cyclophosphamide and PD-L1 simultaneously blockade improved the survival of the treated mice (FIG. 13B).

The frequencies of the CD4+ Tregs and CD8⁺ TILs in the tumor upon treatment were also investigated. Free cyclophosphamide and cyclophosphamide-loaded platelets selectively depleted Tregs within the tumor (FIG. 13C and FIG. 14B) and increased the frequency of Ki67⁺ T cells (FIG. 13D, 13E). Of note, despite PD-1-expressing platelets had limited effect in reducing Tregs, they still increased the frequency of Ki67⁺ T cells (FIG. 13D, 13E). Remarkably, the frequency of CD8⁺ TILs was significantly increased in tumors collected from mice treated with cyclophosphamide-loaded PD-1-expressing platelets (FIG. 13F, 13G), and these cells showed GzmB expression (FIG. 13H, 13I). Immunofluorescence staining also revealed enhanced density of infiltrated CD8⁺ T cell in the mice treated with cyclophosphamide-loaded PD-1-expressing platelets as compared to control mice (FIG. 13J, 13K). Mice treated with low dose cyclophosphamide, and cyclophosphamide-loaded platelets showed delayed hair growth in the abdomen and slighted weight loss (FIG. 14A, 14C). These results demonstrated that the combined utilization of PD-1-expressing platelets and cyclophosphamide effectively disrupted the immune blockade of PD-L1 and depleted the Tregs, leading to the reduced tumor relapse rate after surgery.

### b) Conclusions

In summary, platelets presenting PD-1 were genetically engineered, which can accumulate in surgical wound sites and blockade PD-L1 on the residual tumor cells, intensively reverting the exhausted CD8⁺ T cells to eradicate the residual tumor cells. Megakaryocytes progenitor cell cells were engineered to express mouse PD-1, and were induced to produce platelets presenting PD-1. Besides blockading PD-L1, PD-1 platelets also can function as a platform and combine with other immune blockade inhibitors to improve the response rate. Cyclophosphamide-loaded PD-1 platelets formulation disrupted the immune blockade of PD-L1 and Treg cells, which significantly increased the frequency of reinvigorated CD8⁺Ki67⁺GrzmB⁺ lymphocyte cells in the surgical tumor microenvironment. Reinvigorated CD8⁺ eradicated the residual tumor cells and reduced the rate of tumor relapse after surgery.

### c) Methods

### (1) Chemical and Regents

Cyclophosphamide, Thrombin, Wright-Giemsa solution and phosphatase inhibitor cocktail were ordered from Sigma-Aldrich. PD-1 antibody was from Thermo Scientific. PD-L1 antibody was from Sigma-Aldrich. Mouse CD41a (ab63983) and CD42a (ab173503) antibodies were from Abcam. p-selection (sc-8419) was from Santa Cruz biotechnology. Mouse GPVI (MAB6758) antibody was from R&D Systems. Mouse CD4 and CD8 antibodies for immunofluorescent were ordered from Abcam. Staining antibodies included CD3, CD4, CD8, Ki67, Foxp3 for FACS analysis were order from Biolegend Inc. Wheat Germ Agglutinin (WGA) Alexa Fluor 488 and 594 dyes were ordered from purchased from Thermo Scientific.

### (2) Cell Culture

HEK293T were cultured in Dulbecco's Modified Eagle's Medium (DMEM) supplemented with 10% Fetal Bovine Serum (FBS). Mouse megakaryocyte cell line L8057 were kindly provide by professor Alan Cantor at Boston Children's Hospital and Dana-Farber Cancer Institute and were cultured in RPMI 1640 with 20% FBS. The mouse melanoma cell line B16F10 was purchased from the American Type Culture Collection. For bioluminescent in vivo tumor imaging, B16F10-luc cells were gifts from Dr. Leaf Huang at UNC. B16F10 cells were cultured in DMEM supplemented with 10% FBS.

### (3) Plasmid and Stable Cell Line

Lenti vector containing mouse PD-1 with C-terminal monomeric GFP tag (pLenti-C-mGFP-PD-1-puro) and Lenti-vpak packaging kit containing packaging plasmids and transfection reagent were ordered from Origene. HEK293T cells were transiently transfected with the plasmids using transfection reagent from lenti-vpak packaging kit according to the manufacturer's instructions. L8057 cells were infected with the lenti-virus packaged from HEK293T cells and incubated with 6 μg/ml polybrene. After infection for 96 h, L8057 cells were cultured in RPMI 1640 with 20% FBS complementary with 1 μg/ml puromycin to screening the cell lines stable expression of mouse PD-1. After that, the established L8057 cells stable expression mouse EGFP-PD-1 was maintained in 20% FBS complementary with 0.5-1 μg/ml puromycin.

### (4) Prepare Platelets from L8057 Cells

L8057 cells and PD-1 L8057 cells were cultured in RPMI 1640 with 20% FBS. For maturation and differentiate, L8057 cells were stimulated with 100-500 nM PMA for 3 days. Then the cells were cultured for 6 days more to produce proplatelets and platelets. To isolate platelets, the culture medium was centrifuged at 1500 rpm for 20 min to remove the cells. The supernatant was then centrifugation at 12,000 rpm for 20 min at room temperature. The precipitate of the platelets was resuspended carefully in Tyrode's buffer (134 mM NaCl, 12 mM NaHCO₃, 2.9 mM KCl, 0.34 mM Na2HPO4, 1 mM MgCl2, 10 mM HEPES, pH 7.4) or PBS with 1 μM PGE1. To active platelets, 0.5 U thrombin ml-1 were added to the platelet suspension. PGE1 was removed prior to platelet activation.

### (5) Wright-Giemsa Stain

L8057 cells stimulated with 100-500 nM PMA for 3 days. Then the cells were harvested and washed with PBS buffer for three times. After that, the cells were fixed in absolute methanol for 5 min. Cells were stained in Wright-Giemsa Stain Solution for 5 min. The stained cells then were washed with PBS buffer for three times. Finally, the stained cells were observed under microscope with 40×objective.

### (6) Cell Immunofluorescent Assay

L8057 cells stable expression of EGFP-PD-1 were washed with PBS for three times. Then, the cells were fixed with 4% paraformaldehyde for 10 mins. The cells were washed with PBS twice, then incubated with 0.2% Triton X-100 for 5 minutes. Then the cells were blocked with the buffer containing 3% BSA for 1 h. After that CD41a, CD42a and p-selection primary antibodies were incubated with L8057 cells overnight at 4° C., respectively. The cells were washed with PBS for three times. Then the cells were incubated with rhodamine conjugated secondary antibody (KPL) diluted in 1.5% BSA at room temperature in the dark for 1 h. Finally, the nucleus was stained with DAPI for 10 mins. Finally, the cells were washed three times with PBS for 5 min. Confocal microscopy was performed on a FLUO-VIEW laser scanning confocal microscope (Zeiss) in sequential scanning mode using a 63×objective.

### (7) Western Blot

Immunoblotting analysis was performed. For abbreviation, L8057 cells and L8057 cells stable expressing EGFP-PD1 were lysed with RIPA lysis buffer (Thermo Scientific). And then, cell lysates were resolved on 12% SDS-PAGE and analyzed by immunoblotting using PD-1, CD41a, CD42a, p-selection, GPVI and R-actin antibodies, followed by enhanced chemiluminescence (ECL) detection (Thermo Scientific).

### (8) B16F10 Cell Binding Assay

B16F10 cells were seeded in confocal wells. EGFP-PD-1 expressing platelets (˜0.5×10⁸) or free platelets (˜0.5×10⁸) labeled with cy5.5 were added to the culture medium and incubated with the B16F10 cells overnight. Then Wheat Germ Agglutinin (WGA), Alexa Fluor 594 conjugate was added to staining the cell membrane of B16F10 for 10 min. After that, nucleus was stained with Hoechst for 10 min. The cells were washed with PBS for three times. Confocal microscopy was performed on confocal microscope (Zeiss) in sequential scanning mode using a 63×objective.

### (9) Collagen Binding Assay

Collagen type I/III derived from mouse (Bio-Rad) was reconstituted to a concentration of 2.0 mg ml in 0.25% acetic acid. 200 μl of the collagen solution was then added to each well of a 96-well assay plate and incubated overnight at 4C. Prior to the collagen binding study, the plate was blocked with 2% BSA and washed three times with PBS. For the collagen binding study, the platelets were stained with WGA Alexa Fluor 594 for 30 min, and then washed with PBS for three times. Labeled Platelets (˜1×10⁷) were added in to replicate wells of collagen-coated or non-collagen-coated plates. After 30 s of incubation, the plates were washed three times. Retained nanoparticles were then dissolved with 100 μl of DMSO for fluorescence quantification using a TeCan Infinite M200 reader.

For confocal imaging, the collagen solution was added to confocal well and incubated overnight at 4C. (˜1×10⁸). The wells were blocked with 2% BSA and WGA Alexa Fluor 594 labeled platelets were incubated with collagen for 2 min and then washed with PBS for three times. Confocal microscopy was performed on confocal microscope (Zeiss) in sequential scanning mode using a 63×objective.

### (10) Aggregation Assay

Aggregation of platelets was assessed using a spectrophotometric method. The platelets in PBS were loaded into cuvette. 0.5 IU⁻¹ of thrombin (Sigma Aldrich) was added to the platelets as indicated. The cuvettes were immediately placed in a TeCan Infinite M200 reader and monitored for change in absorbance at 650 nm overtime. For confocal imaging, the platelets were labeled with WGA Alexa Fluor 594. Then the platelets were loaded to the confocal well and added with 0.5 IU⁻¹ of thrombin for 30 min. Confocal microscopy was performed on confocal microscope (Zeiss) in sequential scanning mode using a 63×objective.

### (11) Drug Loading and Release

To load cyclophosphamide to platelets, 100 μg purified platelets (˜1×10⁸) and 100 μg of cyclophosphamide, were gently mixed in 1 ml PBS and incubate for 2 h at 37° C. Platelets were then washed with PBS by centrifugation at 12,000 rpm for three times. For electroporation shock method, 100 μg purified platelets (˜1×10⁸) and 100 μg of cyclophosphamide gently mixed in 1 ml electroporation buffer (1.15 mM potassium phosphate pH 7.2, 25 mM potassium chloride, 21% Optiprep) at room temperature. The samples were subjected to electroporation at 300 V and 150 μF in 0.4 cm electroporation cuvettes using a MicroPulser Electro-porator (Bio-Rad, USA). After that, the electroporation cuvettes containing samples were incubated for 30 min for the membrane recovery. Platelets were then washed with PBS by centrifugation at 1,2000 rpm for 3 times. The release of cyclophosphamide from platelets (100 μg/mL) was analyzed in PBS (pH7.2) at different time point (at 1 h, 2 h, 4 h, 8 h, 24 h and 48 h, respectively) at 37° C. The amount of Cyclophosphamide released was analyzed using a UV-vis spectrophotometer at the k max value of 202 nm.

### (12) Circulation

PD-1 platelets and free platelet produced from L8057 cells were labeled by NHS-Cy5.5. Then the platelets were washed with PBS for 3 times. The C57BL/6 mice were injected with 200 μL labeled free platelets (˜2×10⁸) or PD-1 platelets (˜2×10⁸) through tail-vein, respectively. The blood of the mice was collected from the eye socket at different time points (at 2 min, 30 min, 1 h, 2 h, 4 h, 8 h, 24 h and 48 h, respectively) after the injection. Then the fluorescence of the serum was measured.

### (13) Biodistribution

Free platelets and PD-1 platelets produced from L8057 cells were labeled by NHS-Cy5.5 in PBS buffer. Following incubation overnight at 4° C., Cy5.5-labeled platelets were washed with PBS for three times. The melanoma tumor bearing C57BL/6 mice were injected with 200 μL Cy5.5 labeled PD-1 platelets (˜2×10⁸) through tail-vein. The control group was injected with PBS. After 24 h, the mice were euthanized and the cancers and major organs were harvested. Finally, fluorescence imaging results and average radio intensities were recorded using a Xenogen IVIS Spectrum imaging system.

### (14) In Vivo Anti-Tumor Efficacy Study

B16F10 luciferase-tagged B16F10 (1×10⁶) melanoma tumor cells were transplanted into the right flank of C57BL/6 mice. Eight days after tumor inoculation, the tumors volume reach around ˜150 mm³. These tumors were then resected, leaving about 15 mm³ (10%) tumor volume to mimic the residual tumors in the surgical bed. Briefly, animals were anesthetized in an induction chamber using isoflurane (up to 5% for induction; 1-3% for maintenance), and anaesthesia was maintained via a nose cone. The tumor area was clipped and aseptically prepped. Sterile instruments were used to remove approximately 90% of the tumor. The wound was closed using an Autoclip wound closing system. The mice were randomly divided into several groups of eight mice (n=8) as indicated. The mice firstly were intravenously injected with different treatment formulations: PBS, free platelets (˜2×10), PD-1 platelets (˜2×10⁸), cyclophosphamide (20 mg/kg), cyclophosphamide loaded free platelets (˜2×10⁸) or cyclophosphamide loaded PD-1 platelets (˜2×10⁸). Immediately after the injection, the surgery was carried out within 10 min one mouse by one mouse. The tumor burden was monitored via the bioluminescence of the cancer cells. The mice were clipped and shaved using a depilatory cream before imaging. Images were taken using an IVIS Lumina imaging system (Perkin Elmer). Tumor size was measured with a digital calliper. The tumor volume (mm³) was calculated as (long diameter×short diameter2)/2. Animals were euthanized when exhibiting signs of impaired health or when the volume of the tumor exceeded 2 cm³.

### (15) Tissue Immunofluorescent Assay

Tumors were dissected from the mice and snap frozen in optimal cutting medium (O.C.T.). Several micrometer sections were cut using a cryotome and mounted on slides. The frozen tumor sections were incubated in PBS for 15 min to remove the embedding medium. The specimens were blocked with the buffer containing 3% BSA. Subsequently, the specimens incubated with CD4 and CD8 primary antibodies (1:50 in 3% BSA) overnight and then washed three times with PBS for 5 min each. After that the specimens were incubated with TRITC secondary antibody (KPL) diluted in 3% BSA at room temperature in the dark for 1 h. Finally, the nucleus was stained with DAPI, and the tissue was washed three times with PBS for 5 min each. Confocal microscopy was performed on a FLUO-VIEW laser scanning confocal microscope (Zeiss) in sequential scanning mode using a 40×objective.

### (16) Statistical Analysis

All results are expressed as the mean±s.d. or the mean±s.e.m. as indicated. Biological replicates were used in all experiments unless otherwise stated. One-way or two-way analysis of variance (ANOVA) and Tukey post-hoc tests were used when more than two groups were compared (multiple comparisons). Survival benefit was determined using a log-rank test. All statistical analyses were performed using the IBM SPSS statistics 19. The threshold for statistical significance was P<0.05.

### D. REFERENCES

A. D. Fesnak, C. H. June, B. L. Levine, Nat. Rev. Cancer 2016, 16, 566-581.
A. Hoos, Nat. Rev. Drug. Discov. 2016, 15, 235-247.
A. K. Palucka, L. M. Coussens, Cell 2016, 164, 1233-1247
Alvarez-Erviti, L. et al. Delivery of siRNA to the mouse brain by systemic injection of targeted exosomes. Nature biotechnology 29, 341-345, doi:10.1038/nbt.1807 (2011).
Amezquita, R. A. & Kaech, S. M. Immunology: The chronicles of T-cell exhaustion. Nature 543, 190-191, doi:10.1038/nature21508 (2017).
Anselmo, A. C. et al. Platelet-like nanoparticles: mimicking shape, flexibility, and surface biology of platelets to target vascular injuries. ACS nano 8, 11243-11253, doi:10.1021/nn503732m (2014).
B. T. Luk, L. Zhang, J. Control Release 2015, 220, 600-607
Balachandran, V. P. et al. Identification of unique neoantigen qualities in long-term survivors of pancreatic cancer. Nature 551, 512-516, doi:10.1038/nature24462 (2017).
Berd, D. & Mastrangelo, M. J. Effect of low dose cyclophosphamide on the immune system of cancer patients: depletion of CD4+, 2H4+ suppressor-inducer T-cells. Cancer research 48, 1671-1675 (1988).
Boussiotis, V. A. Molecular and Biochemical Aspects of the PD-1 Checkpoint Pathway. The New England journal of medicine 375, 1767-1778, doi:10.1056/NEJMra1514296 (2016).
C. M. Hu, L. Zhang, S. Aryal, C. Cheung, R. H. Fang, Proc. Natl. Acad. Sci. U.S.A 2011, 108, 10980-10985
C. M. Hu, R. H. Fang, K. C. Wang, B. T. Luk, S. Thamphiwatana, D. Dehaini, P. Nguyen, P. Angsantikul, C. H. Wen, A. V. Kroll, C. Carpenter, M. Ramesh, V. Qu, S. H. Patel, J. Zhu, W. Shi, F. M. Hofman, T. C. Chen, W. Gao, K. Zhang, S. Chien, L. Zhang, Nature 2015, 526, 118-121
C. Robert, A. Ribas, J. D. Wolchok, F. S. Hodi, O. Hamid, R. Kefford, J. S. Weber, A. M. Joshua, W. J. Hwu, T. C. Gangadhar, A. Patnaik, R. Dronca, H. Zarour, R. W. Joseph, P. Boasberg, B. Chmielowski, C. Mateus, M. A. Postow, K. Gergich, J. Elassaiss-Schaap, X. N. Li, R. Iannone, S. W. Ebbinghaus, S. P. Kang, A. Daud, Lancet 2014, 384, 1109-1117
C. Robert, G. V. Long, B. Brady, C. Dutriaux, M. Maio, L. Mortier, J. C. Hassel, P. Rutkowski, C. McNeil, E. Kalinka-Warzocha, K. J. Savage, M. M. Hernberg, C. Lebbe, J. Charles, C. Mihalcioiu, V. Chiarion-Sileni, C. Mauch, F. Cognetti, A. Arance, H. Schmidt, D. Schadendorf, H. Gogas, L. Lundgren-Eriksson, C. Horak, B. Sharkey, I. M. Waxman, V. Atkinson, P. A. Ascierto, N. Engl. J. Med. 2015, 372, 320-330.
C. Robert, J. Schachter, G. V. Long, A. Arance, J. J. Grob, L. Mortier, A. Daud, M. S. Carlino, C. McNeil, M. Lotem, J. Larkin, P. Lorigan, B. Neyns, C. U. Blank, O. Hamid, C. Mateus, R. Shapira-Frommer, M. Kosh, H. Zhou, N. Ibrahim, S. Ebbinghaus, A. Ribas, N. Engl. J. Med. 2015, 372, 2521-2532
C. Wang, W. Sun, Y. Ye, Q. Hu, H. N. Bomba, Z. Gu, Nat. Biomed. Eng. 2017, 1, 0011
C. Wang, Y. Ye, Q. Hu, A. Bellotti, Z. Gu, Adv. Mater. 2017, 29.
D. H. Munn, A. L. Mellor, J. Clin. Invest. 2007, 117, 1147-1154.
Disis, M. L. & Stanton, S. E. Can immunity to breast cancer eliminate residual micrometastases? Clinical cancer research: an official journal of the American Association for Cancer Research 19, 6398-6403, doi:10.1158/1078-0432.CCR-13-0734 (2013).
E. J. Wherry, Nat. Immunol. 2011, 12, 492-499.
E. J. Wherry, S. J. Ha, S. M. Kaech, W. N. Haining, S. Sarkar, V. Kalia, S. Subramaniam, J. N. Blattman, D. L. Barber, R. Ahmed, Immunity 2007, 27, 670-684.
El-Andaloussi, S. et al. Exosome-mediated delivery of siRNA in vitro and in vivo. Nature protocols 7, 2112-2126, doi:10.1038/nprot.2012.131 (2012).
G. Frumento, R. Rotondo, M. Tonetti, G. Damonte, U. Benatti, G. B. Ferrara, J. Exp. Med. 2002, 196, 459-468.
G. M. Lynn, R. Laga, P. A. Darrah, A. S. Ishizuka, A. J. Balaci, A. E. Dulcey, M. Pechar, R. Pola, M. Y. Gerner, A. Yamamoto, C. R. Buechler, K. M. Quinn, M. G. Smelkinson, O. Vanek, R. Cawood, T. Hills, O. Vasalatiy, K. Kastenmuller, J. R. Francica, L. Stutts, J. K. Tom, K. A. Ryu, A. P. Esser-Kahn, T. Etrych, K. D. Fisher, L. W. Seymour, R. A. Seder, Nat. Biotechnol. 2015, 33, 1201-1210
Grossman, W. J. et al. Human T regulatory cells can use the perforin pathway to cause autologous target cell death. Immunity 21, 589-601, doi:10.1016/j.immuni.2004.09.002 (2004).
Gubin, M. M. et al. Checkpoint blockade cancer immunotherapy targets tumor-specific mutant antigens. Nature 515, 577-581, doi:10.1038/nature13988 (2014).
Gulfam, M. et al. Anticancer drug-loaded gliadin nanoparticles induce apoptosis in breast cancer cells. Langmuir: the ACS journal of surfaces and colloids 28, 8216-8223, doi:10.1021/la300691n (2012).
H. Liu, K. D. Moynihan, Y. Zheng, G. L. Szeto, A. V. Li, B. Huang, D. S. Van Egeren, C. Park, D. J. Irvine, Nature 2014, 507, 519-522
H. Zhou, Z. Fan, P. K. Lemons, H. Cheng, Theranostics 2016, 6, 1012-1022.
Hoos, A. Development of immuno-oncology drugs-from CTLA4 to PD1 to the next generations. Nature reviews. Drug discovery 15, 235-247, doi:10.1038/nrd.2015.35 (2016).
Hu, C. M. et al. Nanoparticle biointerfacing by platelet membrane cloaking. Nature 526, 118-121, doi:10.1038/nature15373 (2015).
Hu, Q. et al. Anticancer Platelet-Mimicking Nanovehicles. Adv Mater 27, 7043-7050, doi:10.1002/adma.201503323 (2015).
Huang, A. C. et al. T-cell invigoration to tumor burden ratio associated with anti-PD-1 response. Nature 545, 60-65, doi:10.1038/nature22079 (2017).
I. Mellman, G. Coukos, G. Dranoff, Nature 2011, 480, 480-489 J. Kim, W. A. Li, Y. Choi, S. A. Lewin, C. S. Verbeke, G. Dranoff, D. J. Mooney, Nat. Biotechnol. 2015, 33, 64-72
J. Park, S. H. Wrzesinski, E. Stem, M. Look, J. Criscione, R. Ragheb, S. M. Jay, S. L. Demento, A. Agawu, P. L. Limon, Nat. Mater. 2012, 11, 895-905
J. R. Brahmer, S. S. Tykodi, L. Q. Chow, W. J. Hwu, S. L. Topalian, P. Hwu, C. G. Drake, L. H. Camacho, J. Kauh, K. Odunsi, H. C. Pitot, O. Hamid, S. Bhatia, R. Martins, K. Eaton, S. Chen, T. M. Salay, S. Alaparthy, J. F. Grosso, A. J. Korman, S. M. Parker, S. Agrawal, S. M. Goldberg, D. M. Pardoll, A. Gupta, J. M. Wigginton, N. Engl. J. Med. 2012, 366, 2455-2465
Jang, S. C. et al. Bioinspired exosome-mimetic nanovesicles for targeted delivery of chemotherapeutics to malignant tumors. ACS nano 7, 7698-7710, doi:10.1021/nn402232g (2013).
K. Matsunaga, T. Saitoh, K. Tabata, H. Omori, T. Satoh, N. Kurotori, I. Maejima, K. Shirahama-Noda, T. Ichimura, T. Isobe, S. Akira, T. Noda, T. Yoshimori, Nat. Cell Biol. 2009, 11, 385-396.
Kensler, T. T., Behme, R. J. & Brooke, D. High-performance liquid chromatographic analysis of cyclophosphamide. Journal of pharmaceutical sciences 68, 172-174 (1979).
L. Alvarez-Erviti, Y. Seow, H. Yin, C. Betts, S. Lakhal, M. J. Wood, Nat Biotechnol 2011, 29, 341-345;
L. C. Wu, A. A. Zarrin, Nat. Rev. Immunol. 2014, 14, 247-259.
L. Gu, D. J. Mooney, Nat. Rev. Cancer 2016, 16, 56-66
L. Jeanbart, M. A. Swartz, Proc. Natl. Acad. Sci. U.S.A. 2015, 112, 14467-14472
L. M. Kranz, M. Diken, H. Haas, S. Kreiter, C. Loquai, K. C. Reuter, M. Meng, D. Fritz, F. Vascotto, H. Hefesha, C. Grunwitz, M. Vormehr, Y. Husemann, A. Selmi, A. N. Kuhn, J. Buck, E. Derhovanessian, R. Rae, S. Attig, J. Diekmann, R. A. Jabulowsky, S. Heesch, J. Hassel, P. Langguth, S. Grabbe, C. Huber, O. Tureci, U. Sahin, Nature 2016, 534, 396-401;
L. Riechmann, M. Clark, H. Waldmann, G. Winter, Nature 1988, 332, 323-327
Lefrancais, E. et al. The lung is a site of platelet biogenesis and a reservoir for haematopoietic progenitors. Nature 544, 105-109, doi:10.1038/nature21706 (2017).
M. S. Goldberg, Cell 2015, 161, 201-204
M. T. Stephan, J. J. Moon, S. H. Um, A. Bershteyn, D. J. Irvine, Nat. Med. 2010, 16, 1035-1041;
M. Xie, H. Ye, H. Wang, G. Charpin-El Hamri, C. Lormeau, P. Saxena, J. Stelling, M. Fussenegger, Science 2016, 354, 1296-1301;
Machlus, K. R. & Italiano, J. E., Jr. The incredible journey: From megakaryocyte development to platelet formation. The Journal of cell biology 201, 785-796, doi:10.1083/jcb.201304054 (2013).
Maj, T. et al. Oxidative stress controls regulatory T cell apoptosis and suppressor activity and PD-L1-blockade resistance in tumor. Nature immunology 18, 1332-1341, doi:10.1038/ni.3868 (2017).
Mause, S. F., von Hundelshausen, P., Zernecke, A., Koenen, R. R. & Weber, C. Platelet microparticles: a transcellular delivery system for RANTES promoting monocyte recruitment on endothelium. Arteriosclerosis, thrombosis, and vascular biology 25, 1512-1518, doi:10.1161/01.ATV.0000170133.43608.37 (2005).
Moreau, T. et al. Large-scale production of megakaryocytes from human pluripotent stem cells by chemically defined forward programming. Nature communications 7, 11208, doi:10.1038/ncomms11208 (2016).
O. Hamid, C. Robert, A. Daud, F. S. Hodi, W. J. Hwu, R. Kefford, J. D. Wolchok, P. Hersey, R. W. Joseph, J. S. Weber, R. Dronca, T. C. Gangadhar, A. Patnaik, H. Zarour, A. M. Joshua, K. Gergich, J. Elassaiss-Schaap, A. Algazi, C. Mateus, P. Boasberg, P. C. Tumeh, B. Chmielowski, S. W. Ebbinghaus, X. N. Li, S. P. Kang, A. Ribas, N. Engl. J. Med. 2013, 369, 134-144
Ott, P. A. et al. An immunogenic personal neoantigen vaccine for patients with melanoma. Nature 547, 217-221, doi:10.1038/nature22991 (2017).
P. C. Tumeh, C. L. Harview, J. H. Yearley, I. P. Shintaku, E. J. Taylor, L. Robert, B. Chmielowski, M. Spasic, G. Henry, V. Ciobanu, A. N. West, M. Carmona, C. Kivork, E. Seja, G. Cherry, A. J. Gutierrez, T. R. Grogan, C. Mateus, G. Tomasic, J. A. Glaspy, R. O. Emerson, H. Robins, R. H. Pierce, D. A. Elashoff, C. Robert, A. Ribas, Nature 2014, 515, 568-571
P. Sharma, J. P. Allison, Cell 2015, 161, 205-214
P. Sharma, J. P. Allison, Science 2015, 348, 56-61
P. Vader, E. A. Mol, G. Pasterkamp, R. M. Schiffelers, Adv. Drug. Deliv. Rev. 2016, 106, 148-156
P. Zhang, Y. Chen, Y. Zeng, C. Shen, R. Li, Z. Guo, S. Li, Q. Zheng, C. Chu, Z. Wang, Z. Zheng, R. Tian, S. Ge, X. Zhang, N. S. Xia, G. Liu, X. Chen, Proc. Natl. Acad. Sci. U.S.A. 2015, 112, E6129-6138.
Q. Hu, W. Sun, C. Qian, C. Wang, H. N. Bomba, Z. Gu, Adv. Mater. 2015, 27, 7043-7050.
R. H. Fang, C. M. Hu, B. T. Luk, W. Gao, J. A. Copp, Y. Tai, D. E. O'Connor, L. Zhang, Nano Lett. 2014, 14, 2181-2188;
R. Kuai, L. J. Ochyl, K. S. Bahjat, A. Schwendeman, J. J. Moon, Nat. Mater. 2016.
R. van der Meel, M. H. Fens, P. Vader, W. W. van Solinge, O. Eniola-Adefeso, R. M. Schiffelers, J. Control Release 2014, 195, 72-85
Rollinghoff, M., Starzinski-Powitz, A., Pfizenmaier, K. & Wagner, H. Cyclophosphamide-sensitive T lymphocytes suppress the in vivo generation of antigen-specific cytotoxic T lymphocytes. The Journal of experimental medicine 145, 455-459 (1977).
Ruggeri, Z. M. & Mendolicchio, G. L. Adhesion mechanisms in platelet function. Circulation research 100, 1673-1685, doi:10.1161/01.RES.0000267878.97021.ab (2007).
S. B. Stephan, A. M. Taber, I. Jileaeva, E. P. Pegues, C. L. Sentman, M. T. Stephan, Nat. Biotechnol. 2015, 33, 97-101
S. C. Jang, O. Y. Kim, C. M. Yoon, D. S. Choi, T. Y. Roh, J. Park, J. Nilsson, J. Lotvall, Y. K. Kim, Y. S. Gho, ACS Nano 2013, 7, 7698-7710.
S. El-Andaloussi, Y. Lee, S. Lakhal-Littleton, J. Li, Y. Seow, C. Gardiner, L. Alvarez-Erviti, I. L. Sargent, M. J. Wood, Nat. Protoc. 2012, 7, 2112-2126;
S. K. Schmidt, S. Siepmann, K. Kuhlmann, H. E. Meyer, S. Metzger, S. Pudelko, M. Leineweber, W. Daubener, PLoS One 2012, 7, e44797.
S. L. Topalian, J. M. Taube, R. A. Anders, D. M. Pardoll, Nat. Rev. Cancer 2016, 16, 275-287
S. S. Taneja, J. Urol. 2012, 188, 2148-2149
S. S. Taneja, J. Urol. 2012, 188, 2149.
S. T. Koshy, D. J. Mooney, Curr. Opin. Biotechnol. 2016, 40, 1-8
S. Tan, T. Wu, D. Zhang, Z. Zhang, Theranostics 2015, 5, 863-881
Schumacher, T. N. & Schreiber, R. D. Neoantigens in cancer immunotherapy. Science 348, 69-74, doi:10.1126/science.aaa4971 (2015).
Semple, J. W., Italiano, J. E., Jr. & Freedman, J. Platelets and the immune continuum. Nature reviews. Immunology 11, 264-274, doi:10.1038/nri2956 (2011).
Sharma, P. & Allison, J. P. Immune checkpoint targeting in cancer therapy: toward combination strategies with curative potential. Cell 161, 205-214, doi:10.1016/j.cell.2015.03.030 (2015).
Sharma, P. & Allison, J. P. The future of immune checkpoint therapy. Science 348, 56-61, doi:10.1126/science.aaa8172 (2015).
Sharma, P., Hu-Lieskovan, S., Wargo, J. A. & Ribas, A. Primary, Adaptive, and Acquired Resistance to Cancer Immunotherapy. Cell 168, 707-723, doi:10.1016/j.cell.2017.01.017 (2017).
Siljander, P. R. Platelet-derived microparticles—an updated perspective. Thrombosis research 127 Suppl 2, 530-33, doi:10.1016/0049-3848(10)70152-3 (2011).
Stephan, S. B. et al. Biopolymer implants enhance the efficacy of adoptive T-cell therapy. Nature biotechnology 33, 97-101, doi:10.1038/nbt.3104 (2015).
Stroncek, D. F. & Rebulla, P. Platelet transfusions. Lancet 370, 427-438, doi:10.1016/0140-6736(07)61198-2 (2007).
T. R. Fadel, F. A. Sharp, N. Vudattu, R. Ragheb, J. Garyu, D. Kim, E. P. Hong, N. Li, G. L. Haller, L. D. Pfefferle, S. Justesen, K. C. Herold, T. M. Fahmy, Nat. Nanotechnol. 2014, 9, 723-723
Tian, Y. et al. A doxorubicin delivery platform using engineered natural membrane vesicle exosomes for targeted tumor therapy. Biomaterials 35, 2383-2390, doi:10.1016/j.biomaterials.2013.11.083 (2014).
Tohme, S., Simmons, R. L. & Tsung, A. Surgery for Cancer: A Trigger for Metastases. Cancer research 77, 1548-1552, doi:10.1158/0008-5472.CAN-16-1536 (2017).
Tumeh, P. C. et al. PD-1 blockade induces responses by inhibiting adaptive immune resistance. Nature 515, 568-571, doi:10.1038/nature13954 (2014).
Twyman-Saint Victor, C. et al. Radiation and dual checkpoint blockade activate non-redundant immune mechanisms in cancer. Nature 520, 373-377, doi:10.1038/nature14292 (2015).
von Boehmer, H. Mechanisms of suppression by suppressor T cells. Nature immunology 6, 338-344, doi:10.1038/ni1180 (2005).
W. W. Gibbs, Sci. Am. 2005, 293, 78-83.
W. Zou, J. D. Wolchok, L. Chen, Sci. Transl. Med. 2016, 8, 328rv324.
Y. Tian, S. Li, J. Song, T. Ji, M. Zhu, G. J. Anderson, J. Wei, G. Nie, Biomaterials 2014, 35, 2383-2390.
Y. Ye, J. Wang, Q. Hu, G. M. Hochu, H. Xin, C. Wang, Z. Gu, ACS Nano 2016, 10, 8956-8963.
Yoshida, S., Nomoto, K., Himeno, K. & Takeya, K. Immune response to syngeneic or autologous testicular cells in mice. I. Augmented delayed footpad reaction in cyclophosphamide-treated mice. Clinical and experimental immunology 38, 211-217 (1979).
Zhang, X. et al. The effect of autophagy inhibitors on drug delivery using biodegradable polymer nanoparticles in cancer treatment. Biomaterials 35, 1932-1943, doi:10.1016/j.biomaterials.2013.10.034 (2014).
Zou, W. Regulatory T cells, tumor immunity and immunotherapy. Nat Rev Immunol 6, 295-307, doi:10.1038/nri1806 (2006).
Zou, W., Wolchok, J. D. & Chen, L. PD-L1 (B7-H1) and PD-1 pathway blockade for cancer therapy: Mechanisms, response biomarkers, and combinations. Science translational medicine 8, 328rv324, doi:10.1126/scitranslmed.aad7118 (2016).

1. In one aspect, disclosed herein are engineered nanovesicle or engineered platelet encoding one or more exogenous protein receptors.

2. The engineered nanovesicle or engineered platelet of claim 1, wherein the one or more exogenous protein receptors comprises PD-1, TIGIT, LAG3, or TIM3.

3. The engineered nanovesicle or engineered platelet of claim 1, wherein the nanovesicle is derived from a dendritic cell, stem cell, immune cell, megakaryocyte progenitor cell, or macrophage.

4. A pharmaceutical composition comprising the engineered nanovesicle or engineered platelet of claim 1.

5. The pharmaceutical composition of claim 4, further comprising a therapeutic agent.

6. The pharmaceutical composition of claim 5, wherein the therapeutic agent is encapsulated in the engineered nanovesicle or engineered platelet.

7. The pharmaceutical composition of claim 5, wherein the therapeutic agent is a small molecule, siRNA, peptide, peptide mimetic, or antibody.

8. The pharmaceutical composition of claim 7, wherein the therapeutic agent comprises 1-methyl-tryptophan (1-MT), norharmane, rosmarinic acid, epacadostat, navooximod, doxorubicin, tamoxifen, paclitaxel, vinblastine, or 5-fluorouracil.

9. The pharmaceutical composition of claim 7, wherein the therapeutic agent comprises an anti-PDL-1 antibody.

10. The pharmaceutical composition of claim 9, wherein the antibody is Atexolizumab, Durvalumab, or Avelumab.

11. The pharmaceutical composition of claim 7, wherein the therapeutic agent comprises cyclophosphamide.

12. A method of treating a cancer in a subject comprising administering to a patient with a cancer the engineered nanovesicle or engineered platelet claim 1.

13. The method of claim 12, wherein the cancer comprises melanoma, renal cell carcinoma, non-small cell lung carcinoma, or bladder cancer.

14. The method of treating cancer of claim 12, wherein the engineered nanovesicles, engineered platelets, or pharmaceutical composition are administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

15. The method of treating cancer of claim 12, wherein the engineered nanovesicles, engineered platelets, or pharmaceutical composition are administered at least 1, 2, 3, 4, 5, 6, 7 times per week.

16. The method of treating cancer of claim 12, wherein the dose of the administered engineered nanovesicle, engineered platelets, or pharmaceutical composition is from about 10 mg/kg to about 100 mg/kg.

17. The method of treating cancer of claim 12, further comprising administering a chemotherapeutic agent.

18. The method of treating cancer of claim 12, wherein the engineered nanovesicles, engineered platelets, or pharmaceutical composition are administered following surgical rescission of the tumor.

19. The method of claim 12, wherein the engineered nanovesicle or engineered platelet is administered as a pharmaceutical composition.
